(19) United States
(12) Patent Application Publication
(10) Pub. No.: US 2024/0343841 A1
(43) Pub. Date: Oct. 17, 2024

(54) ENZYME-ASSISTED ATRP PROCEDURES

(71) Applicant: Carnegie Mellon University, Pittsburgh, PA (US)

(72) Inventors: Krzysztof Matyjaszewski, Pittsburgh, PA (US); Alan Enciso Barros, Pittsburgh, PA (US); Liye Fu, Pittsburgh, PA (US); Alan J. Russell, Pittsburgh, PA (US)

(21) Appl. No.: 18/419,836

(22) Filed: Jan. 23, 2024

Related U.S. Application Data
(63) Continuation of application No. 17/942,952, filed on Sep. 12, 2022, now Pat. No. 11,919,991.
Application No. 17/942,952 is a continuation of application No. 16/520,008, filed on Jul. 23, 2019, now Pat. No. 11,472,894.
(60) Provisional application No. 62/764,221, filed on Jul. 23, 2018.

(51) Int. Cl. C08F 4/10; C08F 2/16; C08F 4/26; C08F 220/28; C08K 5/1545; C12P 7/00

(52) U.S. Cl. CPC C08F 4/10; C08F 2/16; C08F 4/26; C08K 5/1545; C12P 7/00; C08F 220/28; C08F 220/286; C08F 2438/01; C12Y 101/03004; C12Y 111/01007

(57) ABSTRACT

Materials and methods for conducting an atom transfer radical polymerization in the presence of oxygen by interlocking enzymatic activities are provided herein.

### CROSS-REFERENCE TO RELATED APPLICATIONS

This application claims priority from U.S. application Ser. No. 17/942,952, filed Sep. 12, 2022, which claim priority from U.S. application Ser. No. 16/520,008, filed Jul. 23, 2019, (now U.S. Pat. No. 11,472,894) which claims priority from U.S. Provisional Application Ser. No. 62/764,221, filed Jul. 23, 2018, which are incorporated herein by reference in their entirety.

### TECHNICAL FIELD

The present invention discloses methods for conducting atom transfer radical polymerization (ATRP) in the presence of oxygen using, for example, coordinated enzymatic activities to remove or utilize oxygen during controlled polymerization procedures.

### BACKGROUND

ATRP is a controlled radical polymerization process that allows effective control over the molecular weight (MW) of the formed polymer, preparation of polymers with narrow molecular weight distributions (dispersity, Ð), incorporation of precisely placed functionalities within the polymer, and fabrication of polymers with various architectures in addition to the synthesis of well-defined composite materials. ATRP is one of the most widely used Controlled Radical Polymerization (CRP) or Reversible-Deactivation Radical Polymerization (RDRP) techniques, due to the range of (co) monomers that can be polymerized and the spectrum of initiators that allow the synthesis of polymers with multiple complex architectures (Tsarevsky and Matyjaszewski, J. Am. Chem. Soc. 2014, 136:6513-6533). Over time, the fundamental ATRP procedure has been expanded to provide diverse experimental procedures that allow for a reduction in the amount of catalyst required for polymerization, and permit the reaction to be conducted in a range of media, including the synthesis of polymers under homogeneous and heterogeneous aqueous conditions (Averick et al., ACS Macro Letters 2012, 1:6-10). This allowed for the use of ATRP to develop an area of research based on the synthesis and modification of biological systems (Pan et al., Angew. Chem. Int. Ed. 2017, 56:2740-2743; Cummings et al., ACS Macro Letters 2016, 5:493-497; Qi et al., Nat. Biomed. Eng. 2016, 1:1-12; and Averick et al., J. Control. Release 2015, 205:45-57).

During the course of an ATRP, there often is an accumulation of the deactivator form of the catalyst as a result of bimolecular termination. This increase in deactivator concentration can change the equilibrium between the dormant and active species, and eventually the polymerization rate can slow or even stop at incomplete conversion if incorrect polymerization conditions are selected. This limitation was initially overcome by adding high concentrations of relatively less active catalysts. More recent developments have overcome this phenomenon through the use of reducing agents that can re-form the activator and allow higher (to almost complete) conversion of monomers to be realized, with much lower concentrations of added active catalyst complexes (Bultz et al., Polymer 2016, 106:313-319.

Historically, industrial scale radical-based polymerizations were performed in anaerobic environments ensuring that air did not penetrate the reaction mixture, as propagating radicals can react with oxygen and form stable peroxy radicals and hydroperoxides that react very slowly with monomers, thus quenching chain growth. Although ATRP displays a degree of robustness that permits the use of protic solvents (Simakova et al., Macromol. 2012, 45:6371-6379), high tolerance to oxygen represents a major challenge, particularly in small scale laboratory polymerizations. Oxygen is a diatomic molecule with two unpaired electrons in separate orbitals in its outer shell. Oxygen captures radicals in a very efficient manner, and thus can stop the propagation of radical polymerizations. Due to this reactivity and its abundance in the atmosphere, oxygen is the most undesired radical scavenger in polymer synthesis. To address this limitation, degassing for long periods of time with inert gasses, use of a glove box or more complicated techniques that are not always scalable (e.g., “freeze, pump, thaw”) are regularly used for the effective removal of oxygen in small scale laboratory procedures. Until now, however, large scale, economic methods that allow the execution of RDRP procedures (including ATRP) in oxygen-rich environments have not been achieved.

### SUMMARY

This document is based, at least in part, on the development of materials and methods that permit efficient and effective ATRP reactions in the presence of oxygen. For example, this document is based, at least in part, on the discovery that fully-oxygen tolerant ATRP can be enabled by the continuous conversion of oxygen to carbon dioxide, catalyzed by the interaction of glucose oxidase (GOx) or another oxidase (e.g., pyranose-2-oxidase; P2Ox) with glucose or another oxidase substrate (e.g., another sugar) in the presence of a reactive oxygen species (ROS) scavenger (e.g., sodium pyruvate or another radical mediator, such as horseradish peroxidase; HRP), as sequential sacrificial substrates that remove the formed hydrogen peroxide (H₂O₂) from the reaction. This document also is based, at least in part, on the discovery that “oxygen fueled” enzymatic ATRP can be driven by consumption of oxygen to enzymatically generate radicals in situ and initiate an enzymatic-Initiators for Continuous Activator Regeneration-(ICAR-) ATRP. Further, this document is based, at least in part, on the discovery that nano-molar concentrations of enzymes and ppm amounts of Cu in a vessel completely open to air under physiologically relevant conditions can provide excellent control over the polymerization of biocompatible monomers, generating bioconjugates linked to polymer segments with controlled high molecular weight and low Ð in less than an hour. In addition, this document is based, at least in part, on the discovery that surface-initiated ATRP (SI-ATRP) can be conducted from substrates of any topology in an open vessel in the presence of GOx, glucose, and a ROS scavenger.

Thus, in some embodiments, this document provides methods for oxygen-tolerant ATRP that include using GOx and glucose to convert oxygen to carbon dioxide in the presence of a ROS scavenger (e.g., sodium pyruvate) or another radical mediator (e.g., HRP) to remove hydrogen peroxide from the reaction. In some embodiments, this document also provides “oxygen fueled” enzymatic ATRP methods in which the reaction is driven by consumption of oxygen to enzymatically generate radicals in situ and initiate enzymatic-ICAR-ATRP. In some embodiments, this document provides ATRP methods that include the use of nano-molar concentrations of enzymes and ppm amounts of Cu in a vessel that is open to air under physiologically relevant conditions. Further, in some embodiments, this document provides SI-ATRP methods conducted from a solid substrate in an open vessel in the presence of GOx, glucose, and a ROS scavenger.

In a first aspect, this document features a method for polymerizing free radically copolymerizable monomers. The method can include combining (a) a deoxygenated polymerization medium containing (i) a transition metal catalyst or metal-free organic complex that can participate in a redox reaction, and (ii) an initiator including one or more redox transferable atoms or groups; (b) an oxygen scavenger; and (c) a ROS scavenger. The polymerization medium can be an aqueous medium. The method can be carried out in a reactor with a limited head space comprising air. The method can be carried out in a reactor open to the atmosphere. The oxygen scavenger can include glucose and GOx. The ROS scavenger can include one or more of pyruvate, horseradish peroxidase, an α-keto-acid, catalase, and a catalase-like enzyme selected from the group consisting of ascorbate peroxidase, cytochrome C peroxidase, haloperoxidase, hemoprotein, glutathione peroxidase, glucose oxidase, laccase, monoamine oxidase, cytochrome P450 oxidase, NADPH oxidase, xanthine oxidase, L-gulonolactone oxidase, and superoxide dismutase. In some cases, the oxygen scavenger can include glucose and GOx, and the ROS scavenger can include one or more of pyruvate, horseradish peroxidase, an α-keto-acid, catalase, and a catalase-like enzyme selected from the group consisting of ascorbate peroxidase, cytochrome C peroxidase, haloperoxidase, hemoprotein, glutathione peroxidase, glucose oxidase, laccase, monoamine oxidase, cytochrome P450 oxidase, NADPH oxidase, xanthine oxidase, L-gulonolactone oxidase, and superoxide dismutase. The initiator can be a bioresponsive molecule with one or more site specific functional initiators. The bio-responsive molecule can be a protein, polypeptide, polynucleotide, aptamer, nucleic acid, or other biomolecule that is incorporated into the formed conjugate with the polymer grown from the site-specific incorporated initiator site(s). The method can include transition metal-mediated controlled polymerizing of vinyl monomers in an aqueous based polymerization medium. The method can be an ICAR-ATRP, and a free radical source can be generated in situ by reaction of an enzyme with the product of the oxygen scavenging reaction. The method can be a photo-ATRP, where a free radical source is generated in situ by photo-based reaction of a reducing agent, and where oxygen is removed by reaction of an enzyme with the product of the oxygen scavenging reaction. The method can be an activator regenerated by electron transfer (ARGET) ATRP, where a free radical source is generated in situ by reaction of a reducing agent, and where oxygen is removed by reaction of an enzyme with the product of the oxygen scavenging reaction. The method can be a surface-initiated polymerization. The oxygen scavenger can include glucose and GOx, where the GOx is immobilized on a solid substrate (e.g., glass, metal, or plastic).

In another aspect, this document features an oxygen-driven ATRP method. The method can include combining (a) a deoxygenated polymerization medium containing (i) copper at a concentration less than 1500 ppm, wherein the copper is capable of participating in a redox reaction, and (ii) a halogen-containing initiator having one or more redox transferable atoms or groups; (b) an oxygen scavenger comprising glucose and GOx; and (c) a ROS scavenger, where the ROS scavenger can be a pyruvate, horseradish peroxidase, an α-keto-acid, a catalase, or a catalase-like enzyme, and where the concentrations of the oxygen scavenger and the ROS scavenger are sufficient to provide a continuous source of radicals for controlled polymerization from the halogen-containing initiator. The polymerization medium can be an aqueous medium. The method can be carried out in a reactor with a limited head space comprising air. The method can be carried out in a reactor open to the atmosphere. The ROS scavenger can include a catalase-like enzyme selected from the group consisting of ascorbate peroxidase, cytochrome C peroxidase, haloperoxidase, hemoprotein, glutathione peroxidase, glucose oxidase, laccase, monoamine oxidase, cytochrome P450 oxidase, NADPH oxidase, xanthine oxidase, L-gulonolactone oxidase, and superoxide dismutase. The GOx can be immobilized on a solid substrate (e.g., glass, metal, or plastic).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

The figures assist in clarifying certain embodiments of the invention but are not intended to limit the invention to the examples described in the figures, as other monomers and other ratios of reagents, and indeed other reagents, can be employed in the disclosed oxygen tolerant, enzymatically activated polymerization methods.

FIG. 1 is a scheme showing a generally accepted mechanism for ATRP.

FIG. 2 is a scheme illustrating the reaction of GOx with oxygen.

FIG. 3 is a scheme showing the use of GOx-catalyzed oxygen removal in an ICAR ATRP.

FIG. 4 is a scheme illustrating aerobic respiration using adenosine diphosphate (ADP), illustrating the consumption of O₂ present in the reaction medium and flask and the release of CO₂.

FIG. 5 is a scheme showing oxygen tolerant ICAR-ATRP.

FIG. 6 is a scheme showing a procedure for grafting from BSA followed by polymer cleavage.

FIG. 7 is a scheme illustrating oxygen-fueled ATRP driven by the use of glucose, GOx, horseradish peroxidase (HRP), and acetylacetonate (ACAC) enzymatic cascade to continuously generate radicals for an ICAR-ATRP from the formed peroxide.

FIG. 8 is a scheme showing polymerization of bioconjugates via ATRP using enzymes.

FIG. 9 is a scheme for a photo-ATRP method.

FIGS. 10A-10C illustrate the kinetics of GOx deoxygenated aqueous ICAR-ATRP polymerization of oligo (ethylene oxide) methyl ether methacrylate, molecular weight 500 (OEOMA₅₀₀). FIG. 10A is a kinetic plot, FIG. 10B is a graph plotting Mₙ and Ð evolution with conversion, and FIG. 10C is a series of GPC curves at various time points.

FIG. 11 is a graph plotting the effect of conducting the polymerization reaction with various concentration of copper.

FIG. 12A is an image showing a reaction carried out in an open 50 ml flask. FIG. 12B is a graph plotting reaction kinetics. FIG. 12C is a graph plotting evolution of the Mₙ and dispersity of the generated polymers.

FIG. 13 is a graph plotting the evolution of measured molecular weight with conversion for a series of runs with 500 ppm copper targeting different degrees of polymerization (DPs) and a comparison with the theoretical molecular weights as molecular weight evolves.

FIG. 14A is a graph plotting the kinetics of a temporally-controlled oxygen-fueled ATRP reaction. FIG. 14B is a graph plotting the evolution of molecular weight and Ð. FIG. 14C is a graph showing GPC traces of samples taken periodically from the polymerization.

FIG. 15 is a schematic illustrating a self-deoxygenated ATRP reaction using ATRP initiator-modified GOx.

FIG. 16 is schematic illustrating the immobilization of GOx on a solid surface.

### DETAILED DESCRIPTION

ATRP is a type of a reversible-deactivation radical polymerization, and is a means of forming a carbon-carbon bond with a transition metal catalyst. ATRP typically employs an alkyl halide (R—X) initiator and a transition metal complex (e.g., a complex of Cu, Fe, Ru, Ni, or Os) as the catalyst. In an ATRP reaction, the dormant species is activated by the transition metal complex to generate radicals via electron transfer. Simultaneously, the transition metal is oxidized to a higher oxidation state. This reversible process rapidly establishes an equilibrium that predominately is shifted to the side with very low radical concentrations. The number of polymer chains is determined by the number of initiators, and each growing chain has the same probability of propagating with monomers to form living/dormant polymer chains (R-Pn-X). As a result, polymers with similar molecular weights and narrow molecular weight distribution can be prepared.

The basic ATRP process and a number of improvements are described elsewhere. See, for example, U.S. Pat. Nos. 5,763,546; 5,807,937; 5,789,487; 5,945,491; 6,111,022; 6,121,371; 6,124,411; 6,162,882; 6,624,262; 6,407,187; 6,512,060; 6,538,091; 6,541,580; 6,624,262; 6,627,314; 6,759,491; 6,790,919; 6,887,962; 7,019,082; 7,049,373; 7,064,166; 7,125,938; 7,157,530; 7,332,550; 7,407,995; 7,572,874; 7,678,869; 7,795,355; 7,825,199; 7,893,173; 7,893,174; 8,252,880; 8,273,823; 8,349,410; 8,367,051; 8,404,788; 8,445,610; 8,816,001; 8,865,795; 8,871,831; 8,962,764; 9,243,274; 9,410,020; 9,447,042; 9,533,297; and 9,644,042; and Publication Nos. 2014/0183055; 2014/0275420; and 2015/0087795, all of which are incorporated herein by reference to provide background and definitions for the present disclosure.

ATRP also is discussed in a number of publications and reviewed in several book chapters. See, e.g., Matyjaszewski and Zia, Chem. Rev. 2001, 101:2921-2990; Qiu et al., Prog. Polym. Sci. 2001, 26:2083-2134; Wang and Matyjaszewski, J. Am. Chem. Soc. 1995, 117:5614-5615; Coessens et al., Prog. Polym. Sci. 2001, 26:337-377; Braunecker and Matyjaszewski, Prog. Polym. Sci. 2007, 32:93-146; Matyjaszewski, Macromol. 2012, 45:4015-4039; Schröder et al., ACS Macro Letters 2012, 1:1037-1040; Matyjaszewski and Tsarevsky, J. Am. Chem. Soc. 2014, 136:6513-6533; and Kamigaito et al., Chem Rev 2001, 101:3689-3746. Indeed, ATRP can control polymer composition, topology, and position of functionalities within a copolymer (Coessens et al., supra; Advances in Polymer Science; Springer Berlin/Heidelberg: 2002, Vol. 159; Gao and Matyjaszewski, Prog. Polym. Sci. 2009, 34:317-350; Blencowe et al., Polymer 2009, 50:5-32; Matyjaszewski, Science 2011, 333:1104-1105; and Polymer Science: A Comprehensive Reference, Matyjaszewski and Martin, Eds., Elsevier: Amsterdam, 2012; pp 377-428).

Monomers and initiators having a variety of functional groups (e.g., allyl, amino, epoxy, hydroxy, and vinyl groups) can be used in ATRP. ATRP has been used to polymerize a wide range of commercially available monomers, including various styrenes, (meth)acrylates, (meth)acrylamides, N-vinylpyrrolidone, acrylonitrile, and vinyl acetate as well as vinyl chloride (Qiu and Matyjaszewski, Macromol. 1997, 30:5643-5648; Matyjaszewski et al, J. Am. Chem. Soc. 1997, 119:674-680; Teodorescu and Matyjaszewski, Macromol. 1999, 32:4826-4831; Debuigne et al., Macromol. 2005, 38:9488-9496; Lu et al., Polymer 2007, 48:2835-2842; Wever et al., Macromol. 2012, 45:4040-4045; and Fantin et al., J. Am. Chem. Soc. 2016, 138:7216-7219). Non-limiting examples of particular monomers that can be used in ATRP reactions include (oligo (ethylene glycol) methacrylate) (POEGMA), poly(2-dimethylaminoethyl methacrylate) (PDMAEMA), poly(sulfobetaine methacrylate) (PSBMA), poly(2-(methylsulfinyl)ethyl acrylate (PMSEA), oligo (ethylene oxide) methyl ether methacrylate (OEOMA), and (hydroxyethyl) methacrylate (HEMA).

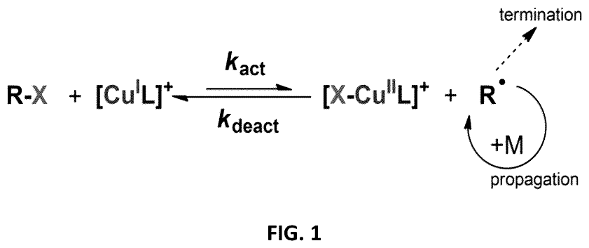

A scheme for a generally accepted ATRP mechanism is shown in FIG. 1. In a classic ATRP, a copper-amine complex in its lower oxidation state (a Cu^{I}L⁺ complex) activates a low fraction of an added alkyl halide initiator (R—X) or a dormant chain end (Pn-X), forming a propagating radical and the deactivator catalyst complex in the higher oxidation state, X-Cu^{II}L⁺. After the addition of a few monomer units, the growing radicals are reverted back to their dormant state (Pn-X), hence minimizing termination reactions and providing concurrent growth of all chains. More recent developments include metal-free ATRP procedures (Jockusch and Yagci, Polymer Chemistry 2016, 10; 1039/c6py01410k), which also can be applied to metal free photo-ATRP (Matyjaszewski, Abstracts of Papers, March 2018 ACS Meeting, POLY-157; and Li et. al., ACS Energy Letters 2018, 3:20-27).

Enzymes are the most potent biological catalysts. One of their main functions in the body is the assembly of monomers (e.g., nucleic bases, saccharides and amino-acids) into biopolymers such as DNA, carbohydrates and proteins. This assembly is initiated by a signal or stimulus (air, light, heat, stress, etc.) that triggers the action of enzymatic interactions through the cells until the demand for the desired bio-polymer is satisfied. One class of enzymes that can activate biological pathways includes copper-containing metalloenzymes that serve as oxido-reductases (Boal and Rosenzweig, Chemical Reviews 2009, 109 (10): 4760-4779), such as laccase, cytochrome c, and superoxide dismutase, or copper transporters including copper chaperones (Davis and O'Halloran, Nature Chem. Bio. 2008, 4 (3): 148-151) and transmembrane proteins (Robinson and Winge, Ann. Rev. Biochem. 2010, 79 (1): 537-562) designed to bring copper from one protein to another or across the cell membrane. Enzymes such as catalase can convert hydrogen peroxide into water and oxygen, as can pyruvic acid and other α-keto-acids (Asmus et al., J. Phys. Chem. A. 2015, 119:966-977).

GOx can be an effective scavenger for oxygen in free radical polymerization (FRP) (Oytun et al., J. Polym. Sci. Part A, Polym. Chem. 2013, 51:1685-1689) and in Enzyme-assisted Reversible Addition-Fragmentation chain Transfer (Enz-RAFT) polymerization (Chapman et al., Angew. Chem. Int. Ed. 2016, 55:4500-4503; Chapman et al., Macromol. 2014, 47:8541-8547; and Gormley et al., Nano Letters 2014, 14:6368-6373). The feasibility of polymerization in open air in the presence of GOx also has been demonstrated (Lv et al., J. Polym. Sci. Part A, Polym. Chem. 2017, 55:164-174; and Tan et al., Macromolecules 2017, 50:5798-5806). In these reports, however, where the generation of extra radicals was not intended (Iwata et al., J. Polym. Sci. Part A, Polym. Chem. 1991, 29:1217-1218), the production of hydrogen peroxide as a side product was ignored even though hydrogen peroxide had been reported to remove the Reversible Addition-Fragmentation chain Transfer (RAFT) end groups from polymers (Jesson et al., Macromolecules 2017, 50:182-191).

Hydrogen peroxide is not a side product that can be ignored in an ATRP, as it is well documented that many transition metals, including those required as catalysts for ATRP, undergo Fenton-like reactions (Pham et al., J. Catalysis 2013, 301:54-64) in the presence of hydrogen peroxide (Helsel and Franz, Dalton Transactions 2015, 44:8760-8770; Shan et al., Chem. Commun. 2016, 52:2087-2090; and Lee et al., Environ. Sci. Technol. 2016, 50:8231-8238), thereby generating many more radical initiators than those accounted for in the planned reaction. This phenomenon precludes controlled polymerization with the ability to prepare polymers having predetermined α-functionality and targeted molecular weight with narrow dispersity.

As disclosed herein, GOx can inexpensively degas solution phase polymerization reactions enzymatically by consuming oxygen as it oxidizes glucose. The examples discussed herein, in which GOx was the only added enzyme, confirmed that generated hydrogen peroxide resulted in poor control over an ATRP, which is in contrast to the reports for FRP and RAFT polymerizations cited above. The methods provided herein extend the ability to use GOx for oxygen removal from an ATRP, as they utilize reaction conditions that avoid any reaction between hydrogen peroxide and the frequently employed copper- or iron-based catalysts.

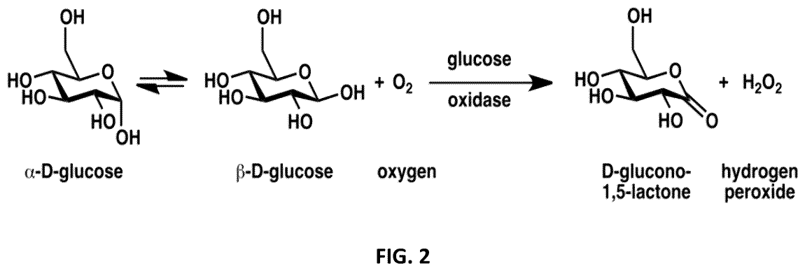

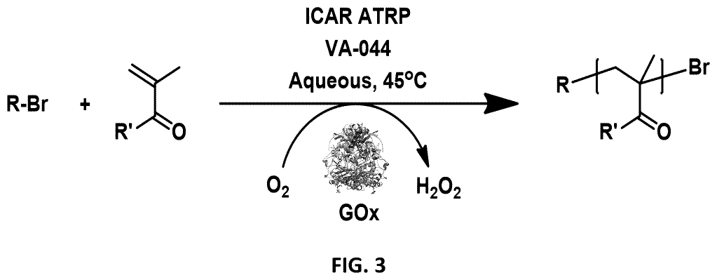

For example, to address the generation of free radicals due to the use of GOx-catalyzed oxygen removal through reaction with glucose (FIG. 2), an initial series of experiments was conducted under ICAR-ATRP conditions (FIG. 3). In these studies, the generated peroxide radicals could act to reactive any formed X—Cu^{II}/L complex, reducing it to an activator Cu^{I}/L complex with concurrent formation of a new ATRP initiator, R—X.

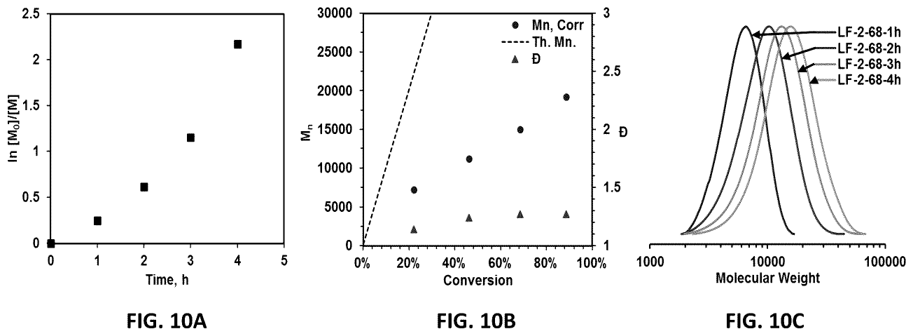

FIGS. 10A-10C and Example 1A show the results of polymerization of two water-soluble, biocompatible monomers, OEOMA₅₀₀ and HEMA. Both reactions showed increased conversion with time and increasing MW with conversion. The reactions were conducted with 1000 ppm of Cu and a target DPₙ=200. After 4 hours, conversion reached 88%, but the experimental molecular weight (M_{n, exp}) was much lower than the theoretical molecular weight (M_{n, Th}) even though the dispersity was acceptable. The results are presented in TABLE 1. Various polymerization parameters were examined, and it was concluded that a significant number of new chains were being generated from a Fenton like reaction between the Cu(I) complex and the hydrogen peroxide product of the oxygen scavenging reaction.

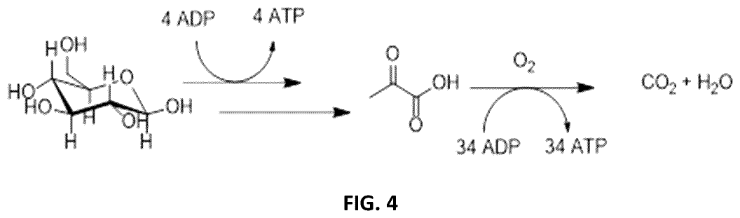

This obstacle was resolved by developing a novel approach to almost immediately remove the hydrogen peroxide from the reaction medium prior to its interaction with the copper catalyst. The approach taken was an aerobic respiration-like process using glucose and oxygen as starting materials that, after a cycle of several enzymatic reactions, results in electron transport and formation of CO₂ and adenosine triphosphate (ATP; FIG. 4). In the aerobic respiration cycle, glucose goes through a series of glycolytic transformations to yield pyruvate and ATP. Pyruvate then reacts with oxygen to generate acetyl-CoA that in the Krebs cycle becomes CO₂ and forms more ATP molecules. Pyruvate is an excellent hydrogen peroxide scavenger in the human body; the reaction of sodium pyruvate with hydrogen peroxide is fast and efficient. Experiments were conducted with GOx and sodium pyruvate, which through a cascade of reactions consumed O₂ present in the reaction medium and flask, releasing CO₂.

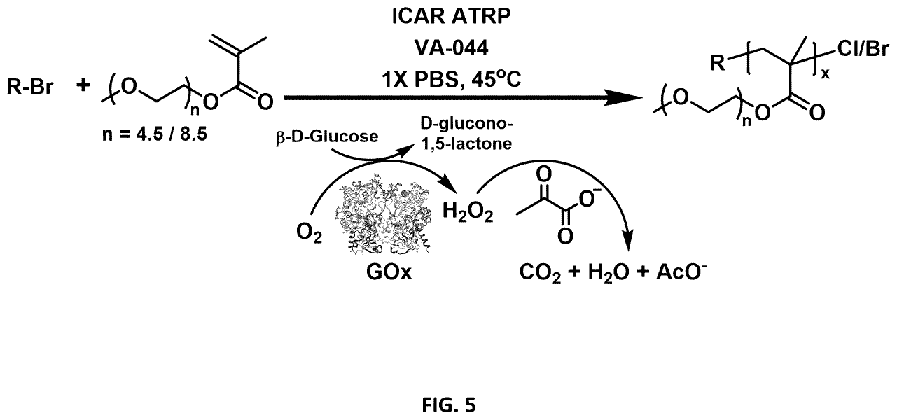

In some embodiments of the polymerization system disclosed herein, glucose and oxygen are catalyzed by GOx into D-glucono-1,5-lactone and hydrogen peroxide, allowing for radical propagation in the presence of air. The hydrogen peroxide then can react extremely quickly with pyruvate to yield CO₂+acetate+water. Taking this procedure into consideration results in the mechanism shown in FIG. 5 for the exemplary aqueous ICAR-ATRP of various methacrylate monomers. In addition to being a potential “green approach” for the synthesis of polymers in the presence of air, the products of the enzymatic elimination of oxygen can be considered commodity chemicals. For example, acetates frequently are used as a feedstock in the chemical industry, and the need for metal acetates is millions of tons annually. In addition, since CO₂ is denser than air, it can provide a protective layer between the reaction mixture and the atmosphere, thereby providing an oxygen insulating layer at the surface of the polymerization.

This system, which included using a combined enzymatic cascade, allowed the controlled polymerization of OEOMA₅₀₀ via aqueous ICAR-ATRP at different targeted DPs, confirming that hydrogen peroxide was successfully removed from the reaction. Polymers with narrow dispersity (1.09≥Ð≤1.2) were obtained, even when high DPs were targeted. After the procedure was optimized, well controlled polymerizations with a good match between theoretical Mₙ and Mₙ measured by corrected GPC (M_{nGPC}) were obtained (TABLE 2). Successful chain extension by the direct addition of OEOMA₃₀₀ monomers to the vial after the first block reached ˜85% conversion, monitored by NMR, demonstrated the retention of chain end functionality and hence the control of the polymerization with low dispersity (Ð=1.3).

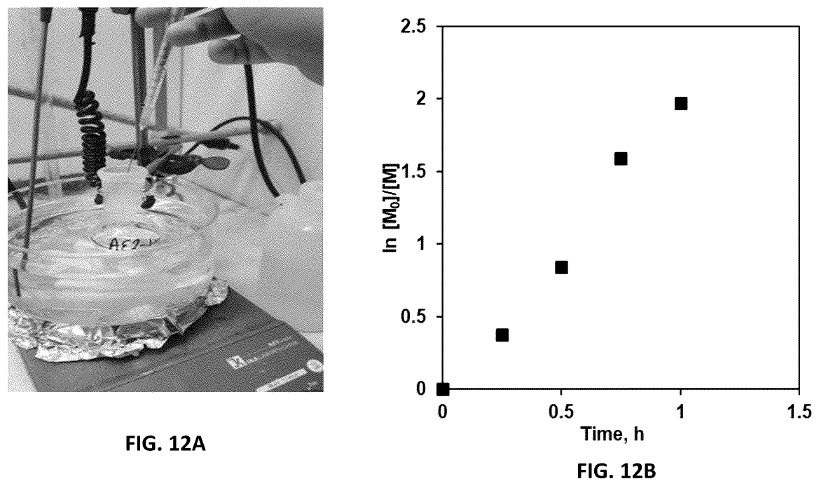

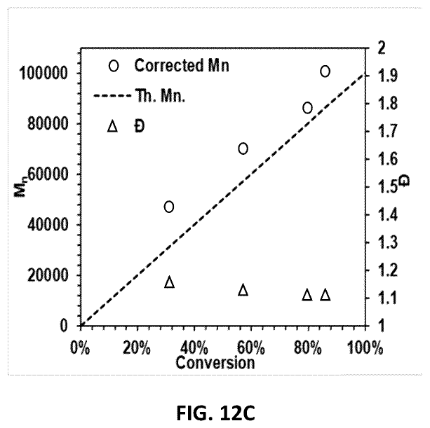

The developed conditions resulted in robust, broadly applicable procedures, indicating that larger scale polymerizations can readily be carried out. The fact that controlled ATRP reactions were accomplished in open air vials indicated that the polymerization conditions were scalable to larger volumes. This was exemplified in the laboratory by using larger containers (50 mL), which provided polymers with low Ð and molecular weights identical to the theoretical targets in the model reactions, FIG. 12.

Any appropriate concentrations of GOx (or other oxidase), glucose (or other oxidase substrate), ROS scavenger can be used in the methods provided herein. In general, the concentrations of these components can be sufficient to provide a continuous source of radicals for controlled polymerization from the initiator. In some cases, suitable concentrations of oxidase (e.g., GOx) can range from about 1 nM to about 500 mM (e.g., about 1 nM to about 10 nM, about 10 nM to about 100 nM, about 100 nM to about 500 nM, about 500 nM to about 1 μM, about 1 μM to about 10 μM, about 10 μM to about 100 μM, about 100 μM to about 1 mM, about 1 mM to about 10 mM, about 10 mM to about 100 mM, or about 100 mM to about 500 mM). In some cases, suitable concentrations of oxidase substrate (e.g., glucose) can range from about 1 mM to about 1 M (e.g., about 1 mM to about 10 mM, about 10 mM to about 100 mM, about 100 mM to about 300 mM, about 200 mM to about 400 mM, about 300 mM to about 500 mM, or about 500 mM to about 1 M). In some cases, suitable concentrations of ROS scavenger can range from about 100 M to about 1 M (e.g., about 100 μM to about 1 mM, about 1 mM to about 10 mM, about 10 mM to about 100 mM, about 100 mM to about 300 mM, about 200 mM to about 400 mM, about 300 mM to about 500 mM, or about 500 mM to about 1 M).

In order to probe the feasible modification of biological systems using this novel enzymatic cascade model, the synthesis of protein-polymer biohybrids was examined via “self-promoted” polymerization of GOx to demonstrate that the enzymes remain catalytically active during and after the polymerization procedure. Two different ATRP initiators-one containing HO-EBiB-like moieties and the other containing BPAA-like moieties-were synthesized with cleavable ester functionalities in their structures, and were tethered separately onto the surface of GOx using-NHS chemistry in PBS (Averick et al. 2015, supra). The polymerizations proceeded following air breathing ICAR-ATRP conditions. After the polymerization of OEOMA₅₀₀ monomer was finished, the polymer was cleaved from the enzymatic structure under basic conditions and analyzed by GPC. The dispersity obtained from the cleaved polymers was close to Ð˜1.17, confirming a well-controlled grafting from reaction.

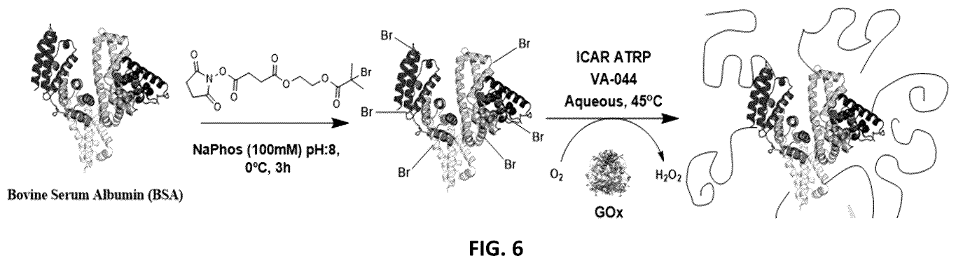

Since the conditions of this enzyme-initiated ATRP are considered biocompatible, a protein-polymer bioconjugate was synthesized using a Bovine Serum Albumin (BSA) based macroinitiator, generated as described elsewhere (Averick et al., supra). The α-bromoisobutyrate (iBBr) groups containing ester tethers were incorporated via NHS-chemistry on the accessible lysine units on the BSA molecules. After the polymerization of OEOMA₅₀₀ using this “oxygen fueled” ATRP system was complete (FIG. 6), the polymer was cleaved from the protein structure under basic conditions (5% NaOH) and analyzed using THF GPC. The results confirmed that the biohybrid (BSA-b-POEOMA) was prepared in a well-controlled manner, providing conjugated polymer chains with Mₙ=63,100 and Ð=1.18. The conditions for the grafting from reaction are shown in TABLE 3. This example confirmed that novel air tolerant ATRP procedure can be applied to the formation of bio-conjugated molecules with well-defined copolymer conjugates.

In addition to pyruvate salt or pyruvic acid, other biocompatible organic and inorganic-based radical scavengers can be used in the methods provided herein. Suitable radical scavengers include, without limitation, thiocarboxylates and other sources of sulfur carboxylate compounds, and catalase-like enzymes. Examples of suitable catalase-like enzymes include, without limitation, ascorbate peroxidase, cytochrome C peroxidase, haloperoxidase, hemoprotein, glutathione peroxidase and the oxidoreductase family—glucose oxidase, laccase, monoamine oxidase, cytochrome P450 oxidase, NADPH oxidase, xanthine oxidase, L-gulonolactone oxidase, and others, such as superoxide dismutase.

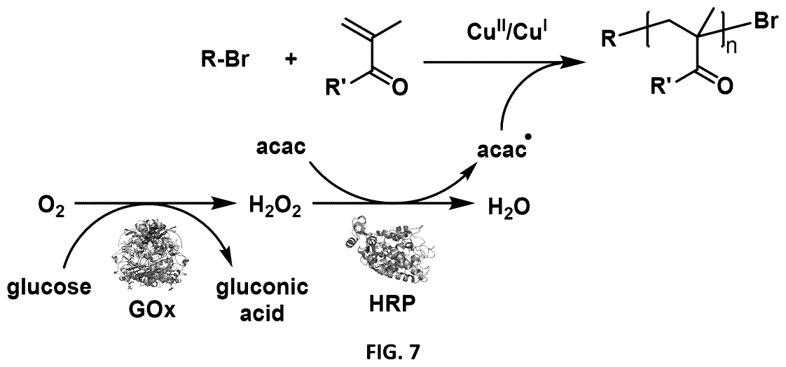

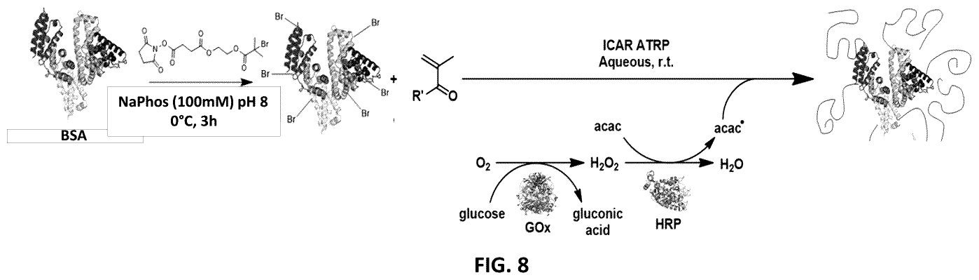

A further extension of the use of biological agents in the oxygen tolerant ATRP process is the use of an enzymatic cascade comprising HRP and ACAC to drive bio-fueled ICAR-ATRP (FIG. 7). This can result in a true enzymatic bio-conjugate self-promoted polymerization (FIG. 8).

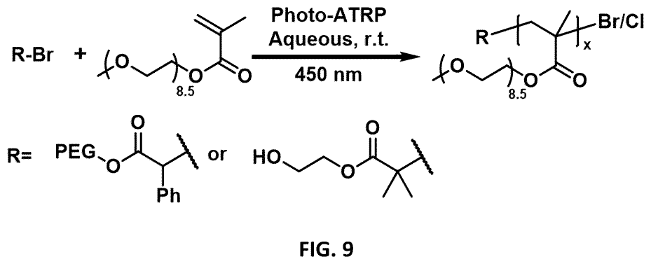

Oxygen-tolerant photo-ATRP methods also are provided herein. Previously, photo-ATRP using blue light irradiation in aqueous media was only achievable in the presence of high Cu loading, 5000-7000 ppm. However when a series of reactions were carried out in the presence of glucose, GOx, and pyruvate (GGP), the reaction attained high conversions (>90%) within 2 hours while the Mₙ and Ð were well controlled using blue light irradiation at 450 nm. A scheme for photo-ATRP is shown in FIG. 9.

The following ratio of reagents were employed in all the reactions summarized in TABLE 12; [Glucose]=200 mM, [GOx]=2 μM, [sodium pyruvate]=100 mM. Total volume=5 mL. All reactions produced polymers with low Ð (the majority lower than 1.25) in high yield, with the majority of products formed with over 90% conversion in 2 hours at copper concentrations as low as to 100 ppm. These aspects of the disclosed methods were exemplified by conducting homogeneous aqueous ICAR-ATRP or photo-ATRP in the presence of oxygen.

In some cases, this document provides ATRP reactivating procedures. For example, surface-initiated ARGET ATRP (SIP) reactions can be carried out using functionalized solid surfaces as initiators to form functionalized particles or functionalized surfaces. GOx-assisted SIP has all the benefits of GOx-assisted solution phase polymerization, enabling the production of polymer brush coatings without degassing. GOx-assistance can makes non-degassed ARGET ATRP far more reliable by reducing variability. In some cases, a monomer such as POEGMA, PDMAEMA, or PMSEA can be grown from a solid surfaces. Oxygen-tolerant ARGET ATRP can be particularly useful for such methods because its oxygen tolerance can be tuned by varying the ratio of reducing agent (e.g., L-ascorbic acid; LAscA) to copper complex. LAscA can reduce Cu(II) species to Cu(I), which can then scavenge oxygen that inhibits polymerization. The amount of LAscA can be chosen to react with both copper and dissolved oxygen, or to reduce only a fraction of the copper.

In any of the methods provided herein, any appropriate amount of transition metal catalyst (e.g., Cu) and reducing agent can be used. In some cases, the concentration of transition metal catalyst can range from about 10 ppm to about 1200 ppm (e.g., about 10 ppm to about 20 ppm, about 20 ppm to about 50 ppm, about 50 ppm to about 100 ppm, about 100 ppm to about 200 ppm, about 200 ppm to about 300 ppm, about 300 ppm to about 500 ppm, about 500 ppm to about 750 ppm, about 750 ppm to about 1000 ppm, or about 1000 ppm to about 1200 ppm). In some cases, suitable amounts of reducing agent can range from about 0.1 μM to about 500 mM (e.g., about 0.1 μM to about 1 μM, about 1 μM to about 10 μM, about 10 μM to about 100 μM, about 100 μM to about 1 mM, about 1 mM to about 10 mM, about 10 mM to about 100 mM, or about 100 mM to about 500 mM). In some cases, the ratio of reducing agent to transition metal catalyst can range from about 0.2:1 to about 20:1 (e.g., about 0.2; 1, about 0.3:1, about 0.4:1, about 0.5:1, about 0.6:1, about 0.7:1, about 0.8:1, about 0.9:1, about 1:1, about 2:1, about 3:1, about 4:1, about 5:1, about 6:1, about 7:1, about 8:1, about 9:1, about 10:1, about 12:1, about 15:1, or about 20:1).

The ability to graft copolymers from the active biological agents utilized in the exemplary reactions discussed above can allow similar reactions to occur in less polar or even organic polymerization media simply by tuning the graft density and graft composition of the biological agents to retain agent activity while allowing solubility in the selected medium comprising the desired monomers.

In some embodiments, this document provides methods resulting from the development of the first fully-oxygen tolerant ATRP, exemplified by utilizing ICAR-ATRP and photo-ATRP, enabled by the continuous conversion of oxygen to carbon dioxide catalyzed by GOx (or another suitable oxidase) with glucose (or another suitable sugar) and a ROS scavenger such as sodium pyruvate or HRP as sequential sacrificial substrates to remove formed hydrogen peroxide, without interaction with either the catalyst or the growing polymerization radical. This procedure was created to eliminate the oxygen present in air in contact with the reaction medium, allowing for grafting of antifouling polymers from solid surfaces, but it also led to development of a new and intriguing approach which can be considered an air stimulated ATRP.

The methods disclosed herein provide open reactors of any scale. These methods can be used to add polymer chains to any appropriate solid surface (e.g., metal, plastic, or glass) or any appropriate molecule. In some cases, the methods provided herein can be used to generate polymer chains on medical device or instrument (e.g., a stent or a hospital instrument), or on a biological molecule such as a protein (e.g., an enzyme such as an esterase, a lipase, an organophosphate hydrolase, a cholinesterase, etc.). Moreover, is noted that the GOx can be added to the reaction medium or, in some cases, can be immobilized on a surface (see, e.g., Nothling et al., Chem. Commun. 2019, 55:8544-8547). The methods disclosed herein also can be used for preparation of functional gels and 3D printing.

As disclosed herein, naturally occurring signals driven by enzymatic cascades and triggered by an oxygen stimulus can be applied to a controlled radical polymerization exemplified by the polymerization of monomers from ATRP imitators, linked to activated amino acids, to create protein-polymer biohybrids. In similar fashion, the chain extension of a DNA strand with methacrylate monomers can be achieved upon exposure to oxygen for the initiation of an enzymatic cascade by oxygen orchestrated by a Cu/tris(2-pyridylmethyl)amine (TPMA) catalyst complex.

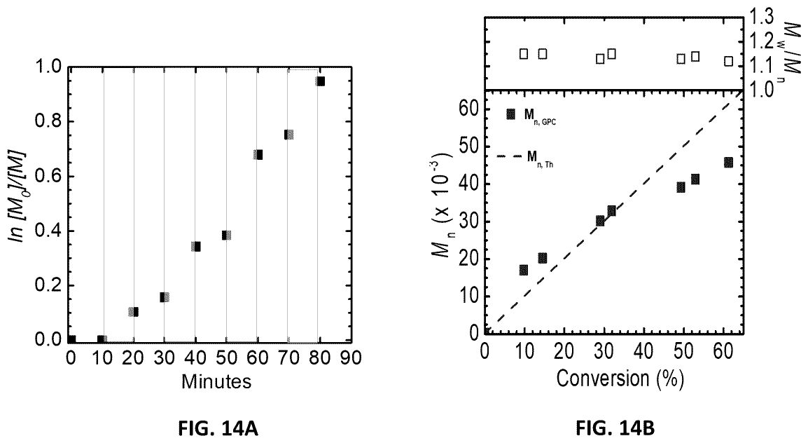

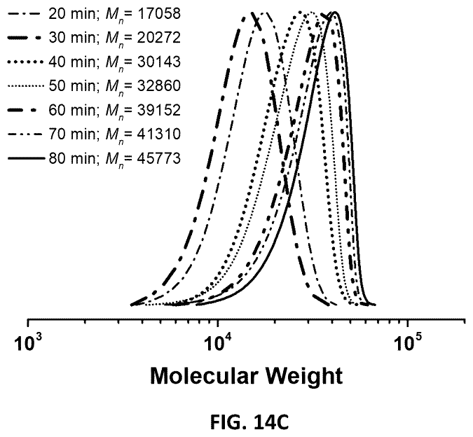

The experimental set up consisted of mixing the varied amounts of substrate and products with GOx, HRP, and ppm amounts of Cu/TPMA catalyst. The sequence of reactions is plotted in FIGS. 14A and 14B, and consists of the catalytic conversion of β-D-glucose and oxygen to hydrogen peroxide and gluconate by GOx. The produced hydrogen peroxide and acetylacetonate (ACAC) reacted with horseradish peroxidase and generated a controlled supply of radicals into the system that reacted with monomers which produced carbon-based radicals, subsequently reducing Cu(II) into Cu(I). A reversible homolytic (pseudo) halogen transfer between a dormant species and the metal complex in the lower state (Cu′X/Ln) then resulted in the formation of propagating polymer chain and a metal complex in a higher oxidation state with a coordinated halide ligand.

Due to the similarities of this procedure to ICAR-ATRP, this approach could be also termed as an enzymatic ICAR-ATRP (enz-ICAR-ATRP). The polymerization experiments reported herein were performed under physiologically relevant conditions and consisted of the use of PBS 1×pH: 6 at 37° C., with a ppm level of Cu/tris(2-pyridylmethyl)amine (TPMA) catalyst. The slight acidity of the reaction medium was selected to ensure optimum working conditions for both enzymes. OEOMA, Mn=500 (OEOMA₅₀₀), was polymerized in a completely open vial using alkyl α-bromophenylacetate as an initiator and 21 mM of β-D-glucose. Cu^{II}Br₂/TPMA was selected as the catalyst since it would form a robust complex that would not undergo a significant disproportionation in aqueous media (Fantin et al., Macromol. 2017, 50 (7): 2696-2705). Since PBS already contained 137 mM of NaCl, the addition of extra salts containing halogens to minimize the dissociation of the anion from the deactivators was not needed (Simakova et al., supra). Well-defined polymers with high molecular weight (Mₙ>70,000) and low dispersities (Ð≤1.13) were obtained in less than one hour under these conditions (FIG. 14C).

GOx and HRP are used commonly in the food industry because of their non-toxicity for humans. The most common uses are for the removal of glucose from egg-whites before drying for use in the baking industry, removal of oxygen from the head-space above bottled and canned drinks and reducing non-enzymatic browning in wines and mayonnaises (Röcker et al., Food Chemistry 2016, 210:660-670; and Bonet et al., Food Chemistry 2006, 99 (2): 408-415). Nevertheless, it was decided to pursue very low concentrations of these enzymes in order not to interfere with the properties of the synthesized polymer. The total amount of protein was decreased to nanomolar (nM) concentrations, representing less than 0.06% (<300 μg) versus the mass of monomer used (500 mg) in the synthesis. In order to understand the role that each one of the links played in this “oxygen fueled” ATRP, their concentrations were modified and the effects on the rate of ATRP, molecular weight and dispersity were measured.

Once the core parameters in this enzymatic machinery were adjusted (TABLE 14) the limits of the control provided by Cu^{II}/TPMA complex at lower ppm concentrations ([Cu]) versus monomer was studied. The results revealed a correlation between the amount of Cu and the M_{w}/Mₙ. Higher concentrations of Cu yielded better control and provided copolymers with lower dispersities, while lower concentrations showed higher Ð and a slower rate of reaction, as expected for ATRP. Nevertheless, using only 100 ppm of catalyst (Entry 12), a polymer with Mₙ=33700 and Ð=1.31 was obtained in 30 minutes. These numbers are significant, considering that the actual concentration of Cu in a 5 mL reaction mixture is only 10 ppm.

Since air was needed to activate the cascade of reactions that produced a continuous flow of radicals, which then reacted with the monomer to produce carbon-based radicals for the (re) generation of Cu^{I} activators, it was expected that polymerization could be switched on/off under aerobic/anaerobic conditions respectively. To verify the need of aerobic environments to induce polymer growth and demonstrate that this system displayed temporal control, an experiment in a Schlenk flask where air was intermittently supplied/removed to/from the reaction mixture. The chain growth stopped after switching to an anaerobic environment. This was achieved by performing a “freeze, pump, thaw” technique, and was restarted after reestablishing an aerobic environment by injecting air to the reaction. Excellent control was achieved over the polymerization and polymers with low dispersity, and predictable molecular weights were prepared. The slow rate of polymerization in the anaerobic (off) steps was attributed to the residual hydrogen peroxide in the solution that radicals generated by HRP, until the substrate was fully consumed. Chain extension was achieved by using a purified macroinitiator based on POEOMA₅₀₀-Br with Mₙ=38,214, Ð=1.13 for the copolymerization of OEOMA₃₀₀, yielding a well-defined block copolymer using this biocatalytic approach, Mₙ=50,765, Ð=1.23.

One main advantage of the procedures disclosed herein over previously known improvements in ATRP, such as procedures that use low concentrations of active catalyst complexes in conjunction with reducing agents, is that one can now remove low concentrations of oxygen from a sealed flask. Another advantage is that the enzymatic cascade of reactions allows ATRP to be conducted in an open reactor. This would be a particular utility if one wanted to run multiple reactions in a well plate reactor, where well defined bioconjugates can be easily prepared in high throughput with simple variation in reagent compositions/concentrations in each well, to prepare evaluate materials for evaluation in a spectrum of medical applications.

The procedure also can be applied to conducting grafting from solid surfaces (e.g., flat or curved metal or plastic surfaces) in “open” continuous flow procedures. Such methods can be used for coating food or beverage containers, metals or coatings for use in ships to reduce/halt biofilm formation, or on the surfaces of instruments for hospitals, for example. In addition, the fact that photo-ATRP can be carried out in the open air means that patterned surfaces can be readily prepared.

The oxygen fueled version of the procedure could be a less expensive method by which to prepare pure polymers or bioconjugates, as low concentrations of catalyst can provide well defined products in a short period of time, thereby allowing high throughput and low catalyst cost in addition to easy purification, with almost no additional reducing agent added to the reaction medium, thereby reducing cost and simplifying the need for post-polymerization purification.

TABLE 1
GOx assisted ICAR-ATRP

| M/I/VA-044/Cu/Ligand | Initiator | [M]/mM | [Cu]/ppm | [NaBr]/mM | time/h | Conv./% | Th•Mn/KD | Mn, GPC | Ð |
|---|---|---|---|---|---|---|---|---|---|
| 200/1/0.3/0.06/0.3 | BPAA | 216 | 300 | 100 | 3 | 95 | 95 | 96.8 | 1.27 |
| 400/1/0.3/0.12/0.6 | BPAA | 216 | 300 | 100 | 2 | 90 | 180 | 110 | 1.23 |
| 600/1/0.3/0.18/0.9 | BPAA | 216 | 300 | 100 | 2 | 50 | 152 | 73.3 | 1.33 |
| 800/1/0.3/0.24/1.2 | BPAA | 216 | 300 | 100 | 2 | 76 | 304 | 67.7 | 1.24 |
| 100/1/0.3/0.03/0.15 | BPAA | 216 | 300 | 100 | 2 | 51 | 25.5 | 23.5 | 1.25 |

TABLE 2
ICAR-ATRP of OEOMA₅₀₀ in the presence of GOx, sodium pyruvate, and oxygen.

| | | [Cu] | time | Conv. | | | Mn_{GPC} | |
|---|---|---|---|---|---|---|---|---|
| No. | M/I/VA-044/Cu/Ligand | (ppm) | (h) | (%) | Mn, Th* | Mn_{GPC} | Corrected* | Ð |
| AE87 | 200/1/0.3/0.1/0.5 | 500 | 1.5 | 96.2 | 96.2 | 91.3 | 142.9 | 1.09 |
| AE88 | 400/1/0.3/0.2/1 | 500 | 1.5 | 98.1 | 196.3 | 144.8 | 233.9 | 1.12 |
| AE89 | 600/1/0.3/0.3/1.5 | 500 | 1.5 | 97.2 | 291.5 | 200.3 | 330.8 | 1.13 |
| AE90 | 800/1/0.3/0.4/2 | 500 | 1.5 | 96.98 | 387.9 | 233.9 | 390.5 | 1.18 |

*Mn expressed in 10⁻³; M = OEOMA₅₀₀, [M] = 10 vol % in PBS, [Br−] = 100 mM, [Glucose] = 200 mM, [Sodium Pyruvate] = 100 mM, [GOx] = 2.5 μM, RXN temperature = 45° C.; DP 200 [I] = 1 mM, DP 400 [I] = 0.5 mM, DP 600 [I] = 0.33 mM, DP 800 [I] = 0.25 mM;
*Corrected Ð, Correction was executed using Mark-Houwink Equation based on PMMA-Toluene as universal calibration.

TABLE 3
Conditions for grafting from BSA polymerization

| | | | [M] | [Cu] | [NaBr] | time | Conv. | Th•Mn | Mn_{GPC} | |
|---|---|---|---|---|---|---|---|---|---|---|
| No. | M/I/VA-044/Cu/Ligand | Initiator | (mM) | (ppm) | (mM) | (h) | (%) | (KD) | Corrected* | Ð |
| 103 | 200/1/0.3/0.2/1 | BiBB | 216 | 1000 | 100 | 1.5 | 92.3 | 92.5 | 81.3 | 1.22 |

*M = OEOMA₅₀₀, [M] = 10 vol % in PBS, [Br−] = 100 mM, [Glucose] = 200 mM, [Sodium Pyruvate] = 100 mM, [GOx] = 2.5 μM, RXN temperature = 45° C.; Correction was executed using Mark-Houwink Equation based on PMMA-Toluene as universal calibration.

The invention will be further described in the following examples, which do not limit the scope of the invention described in the claims.

### EXAMPLES

The first series of experiments were designed based on previous experience with ICAR-ATRP in aqueous systems (Konkolewicz et al., Macromol. 2012, 45:4461-4468), and were adjusted according to the results obtained with the addition of GOx (2.5 μM), NaBr (100 mM) and glucose (200 mM) in environments exposed to air. ICAR-ATRP was selected due to its ability to prevent the sudden oxidation of Cu(I) to Cu(II) through the slow decomposition of VA-044 at contact with air, which in addition to bestowing thermoregulation to the system, offers the capability of turn on/off the reaction once that the oxygen is removed simply by reducing the temperature of the reaction. Interestingly, the first attempts performed in completely opened vials did not yield any polymerization with agitation of the reaction medium, but in the absence of agitation, polymerization was obtained. This situation taught us the importance of the oxygen diffusion in the reaction, which has been reported elsewhere (Lv et al., supra). Subsequently, reactions in a capped vial were executed yielding reproducible polymerizations even with a strong stirring.

The early polymerizations were conducted with a molar ratio of [OEOMA₅₀₀]/[Initiator]/[VA-044]/[Cu]/[TPMA]=200:1:0.3:0.2:0.4 and a volume ratio of monomer/PBS=1:9 at 45° C. The starting polymerization initiator was HO-EBiB, which generated homogeneous chains with relatively low dispersities, Ð˜1.3. However, the molecular weights obtained were far lower than the theoretical values, approximately 4× lower, which made us aware of the possibility that new chains being generated by H₂O₂. For that reason, it was envisioned that a more reactive initiator (Tang and Matyjaszewski, Macromol. 2007, 40:1858-1863) such as α-bromophenyl acetic acid (BPAA), which has an approximately 500× faster K_{ATRP} than HO-EBiB in MeCN, could suppress such side reactions. In fact, while the polymerizations using BPAA showed a better correlation with the theoretical value the dispersity was not significantly improved.

Therefore, a second screening exploring different ratios of reactants was conducted to optimize the polymerization conditions. Experiments with different loadings of catalyst (100, 300, 500 and 1000 ppm) revealed that the amount of Cu can be lowered down to 300 ppm with similar degrees of control; while lower concentrations resulted in broadened dispersity. Varying the ratios of Initiator/VA-044 (1:0.3, 0.1, 0.05, 0.02, 0.01) resulted in the observation that the reaction becomes sluggish and loses control with ratios lower than 1/0.05. Since the reactions were performed in PBS 1×, which contains 137 mM Cl adding extra NaBr salt was not strictly required as reported in other aqueous systems (Konkolewicz et al., supra), although the addition of concentrations >100 mM slowed down the reaction.

During the third stage of the project when higher molecular weights and DPs (>200) were pursued, a new challenge was faced; the obtained experimental Mₙ was again at an increasing distance from the Th. Mₙ, and the polymer chains that were expected to reach 300 kD were as low as 68 kD, almost 5× times lower than the planned molecular weight. This situation showed the limits of tolerance for our initial conditions to the degassing of ambient air by reaction with GOx as the reaction resulted in the continuous generation of H₂O₂, even using a closed vial.

Consequently, this required a search for optimum conditions that could get rid of H₂O₂ and at the same time generate clean side products that did not interfere with an ATRP.

Those requirements made us turn our attention to nature's solutions for intake of oxygen and prompted us to design a previously unreported cascade of reactions that, in a deferential manner, emulate the cycle of transformations of oxygen into CO₂ in living bodies, Scheme 4. The inclusion of sodium pyruvate, as an exemplary peroxide scavenger, to the system was also based recent mechanistic reports that have demonstrated its fast reactivity towards hydrogen peroxide in biological systems (Asmus et al., supra).

Thus, the designed cascade system, emulating the aerobic respiration cycle, did not just decrease the Ð and make the measured Mₙ of the of the polymer chains match the Th. Mₙ, but also allowed high tolerance of oxygen that provided controlled ATRP polymerizations of non-deoxygenated reagents, indeed, even in vessels truly open to air.

Experiments using the improved bio-deoxygenation conditions with different amounts of Cu catalyst (300, 500 and 1000 ppm) at different DPs (200, 400, 600 and 800) showed narrower dispersities (1.09≥Ð≤1.2) and reached quantitative conversions in a maximum of 2 hours.

This oxygen resistant enzymatic ICAR-ATRP system also exhibited different degrees of control over the polymerization depending on the concentration of added catalyst. Higher amounts of catalyst provided better correlation of the Th. Mₙ. for low targeted DPs; however when high DPs were targeted lower amounts of catalyst gave a better correlation of Th. Mₙ with measured Mₙ.

### Example 1—Determining Conditions for Oxygen Tolerant ATRP

### 1A) GOx Plus Glucose Assisted ICAR-ATRP (FIG. 3)

| Exp * | LF-2-68 |
|---|---|
| M:I:VA-044:Cu(II):TPMA | 200; 1:0.3:0.2:0.4 |
| Monomer | OEOMA₅₀₀ |
| Time (h) | 4 |
| Conv. (%) | 88 |
| Mn (kDa) | 19 |
| Ð | 1.27 |

I = HO-EBiB, [M] = 20% (v/v), [NaBr] = 100 mM, [GOx] = 2.5 μM, [Glucose] = 200 mM, solvent = 1X PBS, pH = 7.4

An ATRP reaction was conducted with 1000 ppm of Cu with target DPₙ=200. After 4 hours the conversion reached 88%, but M_{n, exp} was much lower than M_{n, Th} even though the dispersity was acceptable. FIG. 10A shows the kinetics of the polymerization and FIG. 10B shows the relationship between the actual MW and theoretical MW while 1C shows the GPC curves, all displaying narrow Ð.

### 1B) Effect of Catalyst Loading

The second parameter examined was the Cu loading, which was varied from 1000 ppm to down to 100 ppm. As shown in TABLE 4, the polymerization maintained good control down to catalyst concentrations of 300 ppm. When using 100 ppm Cu, the reaction became much slower while the GPC peaks of kinetic samples did not show a significant increase in Mₙ with conversion and dispersity became higher.

TABLE 4
Effect of different concentrations of catalyst

| No. | M/I/VA-044/Cu/Ligand | [Cu]/ppm | time/h | Conv./% | M_{n, Th} | M_{n, GPC} | Ð |
|---|---|---|---|---|---|---|---|
| 3-5a | 200/1/0.3/0.06/0.3 | 300 | 3 | 95 | 95 | 96.8 | 1.27 |
| 3-10 | 200/1/0.3/0.2/1 | 1000 | 1 | 91 | 91 | 97 | 1.18 |
| 3-11 | 200/1/0.3/0.02/0.1 | 100 | 4 | 34 | 34 | 59.6 | 1.52 |
| 3-12 | 200/1/0.3/0.1/0.5 | 500 | 1.5 | 71 | 71 | 66.5 | 1.23 |

M = OEOMA500, [M] = 10 vol %, I = BPAA, [Br⁻] = 100 mM, [Glucose] = 200 mM, [GOx] = 2.5 μM, RXN temperature = 45° C.

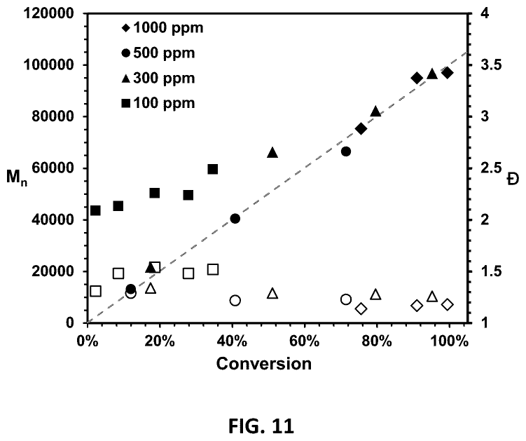

The results are summarized in FIG. 11, which shows the effect of different concentrations of catalyst on the rate of increase in Mₙ with conversion and dispersity.

### 1C) Effect of Targeted DP

The target DPₙ, was varied from DP=800 down to DP=100 with monomer concentrations and Cu loading unchanged, i.e., initiator usage was altered to achieve such changes, as well as corresponding VA-044 usage. It was observed that MW control was not maintained for higher than DP=200. Higher target DP resulted in formation of polymers with Mₙ, GPC much lower than the theoretical value (TABLE 5). However, dispersities of all trials remained reasonably low for all runs with [Cu]=300 ppm or below.

TABLE 5
Effect of targeting different degrees of polymerization

| Run | M/I/VA-044/Cu/Ligand | Target DPn | time/h | Conv./% | M_{n, Th} | M_{n, GPC} | Ð |
|---|---|---|---|---|---|---|---|
| 3-5a | 200/1/0.3/0.06/0.3 | 200 | 3 | 95 | 95 | 96.8 | 1.27 |
| 3-6 | 400/1/0.3/0.12/0.6 | 400 | 2 | 90 | 180 | 110 | 1.23 |
| 3-7 | 600/1/0.3/0.18/0.9 | 600 | 2 | 50 | 152 | 73.3 | 1.33 |
| 3-8 | 800/1/0.3/0.24/1.2 | 800 | 2 | 76 | 304 | 67.7 | 1.24 |
| 3-9 | 100/1/0.3/0.03/0.15 | 100 | 2 | 51 | 25.5 | 23.5 | 1.25 |

M = OEOMA₅₀₀, [M] = 10 vol %, I = BPAA, [Br⁻] = 100 mM, [Glucose] = 200 mM, [GOx] = 2.5 μM, RXN temperature = 45° C.

### 1D) Effect of Changing Initiator to VA-044 Ratio

The next parameter examined was the Initiator: VA-044 ratio. The ratio was varied from 1:0.3 to 1:0.01. The results, presented in TABLE 6, show that the reaction proceeded in a controlled manner from 30% VA-044 to 5%, with the MW matching the theoretical value and narrow dispersity. While decreasing VA-044 portion to lower than 2%, the rate of polymerization became slower and dispersity increased.

TABLE 6
Effect of changing I:VA-044 ratio

| | | | time | Conv. | | | |
|---|---|---|---|---|---|---|---|
| Run | M/I/VA-044/Cu/Ligand | I:VA-044 | (h) | (%) | M_{n, Th} | M_{n, GPC} | Ð |
| 3-5a | 200/1/0.3/0.06/0.3 | 1:0.3 | 3 | 95 | 95.0 | 96.8 | 1.27 |
| 3-5 | 200/1/0.1/0.06/0.3 | 1:0.1 | 3 | 93 | 93.0 | 105.3 | 1.28 |
| 3-20 | 200/1/0.05/0.06/0.3 | 1:0.05 | 3 | 91 | 91.2 | 68.3 | 1.27 |
| 3-21 | 200/1/0.02/0.06/0.3 | 1:0.02 | 3 | 58 | 58.4 | 38.9 | 1.37 |
| 3-22 | 200/1/0.01/0.06/0.3 | 1:0.01 | 3 | 48 | 48.9 | 31.8 | 1.48 |

M = OEOMA₅₀₀, [M] = 10 vol %, I = BPAA, [Br⁻] = 100 mM, [Glucose] = 200 mM,
[GOx] = 2.5 μM, RXN temperature = 45°

### 1E) Effect of Concentration of Added Salt

The last parameter modified was the concentration of the NaBr additive. Since the reaction was always carried out in 1×PBS, the solution contained 137 mM NaCl in all cases. As shown in TABLE 7, even in the case where no NaBr was added, the reaction still proceeded quickly and was well controlled. When increasing amounts of NaBr were added, the reaction rate dropped and the dispersity increased to 1.29.

TABLE 7
Effect of added salt

| | | | | time | Conv. | | | |
|---|---|---|---|---|---|---|---|---|
| Run | M/I/VA-044/Cu/Ligand | [NaBr] | | (h) | (%) | M_{n, Th} | M_{n, GPC} | Ð |
| 3-23 | 200/1/0.3/0.06/0.3 | 0 | mM | 3 | 99 | 99.5 | 109.7 | 1.19 |
| 3-29 | 200/1/0.3/0.06/0.3 | 30 | mM | 3 | 98 | 98.5 | 107.9 | 1.24 |
| 3-5a | 200/1/0.3/0.06/0.3 | 100 | mM | 2 | 95 | 95.3 | 96.8 | 1.27 |
| 3-30 | 200/1/0.3/0.06/0.3 | 200 | mM | 2 | 55 | 55.2 | 41.9 | 1.29 |

M = OEOMA₅₀₀, [M] = 10 vol %, I = BPAA, [Glucose] = 200 mM, [GOx] = 20 μM, RXN temperature = 45° C.

### Example 2—GOx Plus Glucose Assisted ICAR-ATRP

### 2A) In the Absence of a Peroxide Scavenger

TABLE 8 shows the initial results of experiments conducted without the addition of a peroxide scavenger. The results show dispersities (Ðs) that were broader than expected from a well-controlled polymerization, and also demonstrated that when high degrees of polymerization were targeted, the reactions did not reach the targeted molecular weights.

### 2B) Polymerizations Conducted in the Presence of a Peroxide Scavenger

When a peroxide scavenger (pyruvate) was included, the resulting Ðs were narrow and the measured (corrected) Mₙ was closer to theoretical values (TABLE 9).

### 2C) Reactions Conducted Using 500 ppm Copper Targeting Different DPs

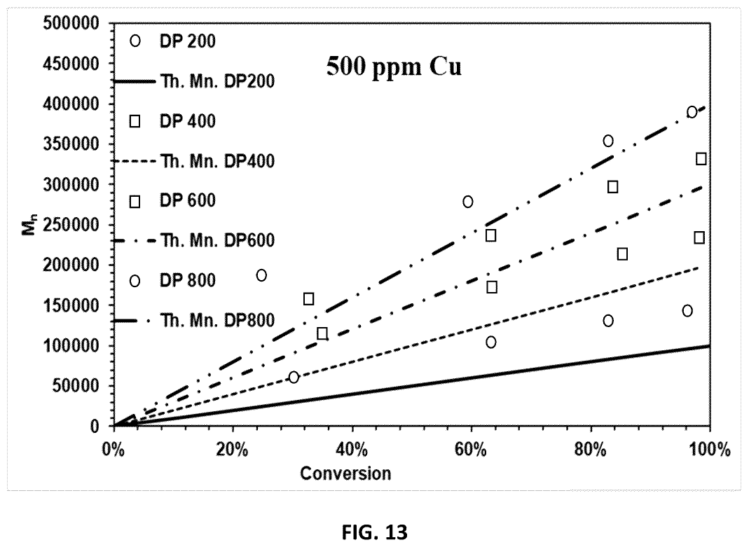

When 500 ppm Cu was included, the polymers resulting from all reactions had narrow molecular weights that were close to theoretical for all targeted DP (TABLE 10). FIG. 13 shows the relationship between targeted DP and rate of polymerization, with lines indicating the evolution of the targeted DPs with conversion.

### Example 3—Oxygen Fueled ICAR-ATRP

The schematic for this reaction is shown in FIG. 7 and the results of a series of experiments examining the role of different concentrations of horseradish peroxidase and different targeted DPs are shown in TABLE 11.

This series of ICAR-ATRP reactions conducted without the addition of a standard free radical initiator resulted in the formation of polymers with molecular weights close to theoretic numbers and low Ð indicating well controlled reactions were carried out.

TABLE 8
GOx + Glucose assisted ICAR-ATRP “non-improved conditions”

| | | [M] | [Cn] | [NaBr] | time | Conv. | Th•Mn | | |
|---|---|---|---|---|---|---|---|---|---|
| M/I/VA-044/Cu/Ligand | Initiator | (mM) | (ppm) | (mM) | (h) | (%) | (KD) | Mn, _{GPC} | Ð |
| 200/1/0.3/0.06/0.3 | BPAA | 216 | 300 | 100 | 3 | 95 | 95 | 96.8 | 1.27 |
| 400/1/0.3/0.12/0.6 | BPAA | 216 | 300 | 100 | 2 | 90 | 180 | 110 | 1.23 |
| 600/1/0.3/0.18/0.9 | BPAA | 216 | 300 | 100 | 2 | 50 | 152 | 73.3 | 1.33 |
| 800/1/0.3/0.24/1.2 | BPAA | 216 | 300 | 100 | 2 | 76 | 304 | 67.7 | 1.24 |
| 100/1/0.3/0.03/0.15 | BPAA | 216 | 300 | 100 | 2 | 51 | 25.5 | 23.5 | 1.25 |

TABLE 9
GOx + Glucose + Pyruvate assisted ICAR-ATRP

| | | | | | | | | Mn _{GPC}, | | |
|---|---|---|---|---|---|---|---|---|---|---|
| | | [M] | [Cu] | [NaBr] | time | Conv. | | before | Mn, _{GPC} | |
| No. | M/I/VA-044/Cu/Ligand | (mM) | (ppm) | (mM) | (h) | (%) | Th•Mn* | correction* | corrected* | Ð |
| 82 | 100/1/0.3/0.03/0.15 | 216 | 300 | 100 | 1.5 | 91.6 | 45.8 | 82.8 | 128.7 | 1.15 |
| 81 | 200/1/0.3/0.06/0.3 | 216 | 300 | 100 | 2 | 95.4 | 95.4 | 110.9 | 175.9 | 1.17 |
| 78 | 400/1/0.3/0.12/0.6 | 216 | 300 | 100 | 1.5 | 97 | 194 | 148.1 | 239.6 | 1.17 |
| 79 | 600/1/0.3/0.18/0.9 | 216 | 300 | 100 | 1.5 | 94.4 | 283.3 | 203.5 | 336.5 | 1.18 |
| 80 | 800/1/0.3/0.24/1.2 | 216 | 300 | 100 | 1.5 | 96 | 384 | 263.2 | 442.9 | 1.27 |
| 83 | 200/1/0.3/0.2/1 | 216 | 1000 | 100 | 1 | 92.8 | 92.8 | 74.4 | 114.8 | 1.13 |
| 84 | 400/1/0.3/0.4/2 | 216 | 1000 | 100 | 1 | 94.23 | 188.5 | 106.7 | 168.8 | 1.17 |
| 85 | 600/1/0.3/0.6/3 | 216 | 1000 | 100 | 1 | 95.12 | 285.4 | 125.3 | 200.4 | 1.18 |
| 86 | 800/1/0.3/0.8/4 | 216 | 1000 | 100 | 1 | 95.1 | 380.3 | 147 | 237.7 | 1.2 |
| 76 | 200/1/0.3/0.1/0.5 | 216 | 500 | 100 | 1 NK | 92.5 | 92.5 | 109.2 | 173 | 1.12 |
| 77 | 500/1/0.3/0.1/0.5 | 216 | 200 | 100 | 2 NK | 99 | 247.5 | 237.5 | 396.9 | 1.17 |

*Mn expressed in 10⁻³; M = OEOMA₅₀₀, [M] = 10 vol %, I = BPAA, [Br⁻] = 100 mM, [Glucose] = 200 mM, [Sodium Pyruvate] = 100 mM, [GOx] = 2.5 μM, RXN temperature=

TABLE 10
Effect of targeting different DPs.

| | | [Cu] | time | Conv. | | | Mn_{GPC} | |
|---|---|---|---|---|---|---|---|---|
| Run | M/I/VA-044/Cu/Ligand | (ppm) | (h) | (%) | Mn, Th* | Mn_{GPC}* | corrected* | Ð |
| AE87 | 200/1/0.3/0.1/0.5 | 500 | 1.5 | 96.2 | 96.2 | 91.3 | 142.9 | 1.09 |
| AE88 | 400/1/0.3/0.2/1 | 500 | 1.5 | 98.1 | 196.3 | 144.8 | 233.9 | 1.12 |
| AE89 | 600/1/0.3/0.3/1.5 | 500 | 1.5 | 97.2 | 291.5 | 200.3 | 330.8 | 1.13 |
| AE90 | 800/1/0.3/0.4/2 | 500 | 1.5 | 96.98 | 387.9 | 233.9 | 390.5 | 1.18 |

*Mn expressed in 10⁻³; M = OEOMA₅₀₀, [M] = 10 vol % in PBS, [Br−] = 100 mM, [Glucose] = 200 mM, [Sodium Pyruvate] = 100 mM, [GOx] = 2.5 μM, RXN temperature = 45° C.; DP 200 [I] = 1 mM, DP 400 [I] = 0.5 mM, DP 600 [I] = 0.33 mM, DP 800 [I] = 0.25 mM; Correction was executed using Mark-Houwink Equation based on PMMA-Toluene as universal calibration.

TABLE 11
ICAR-ATRP controlled by hydrogen peroxide formed during GOx oxygen removal reaction

| | | [M] | HRP | Radical | time | Conv. | | Mn, _{GPC} | |
|---|---|---|---|---|---|---|---|---|---|
| No. | M/I/RI/Cu/TPMA | (mM) | (nM) | Initiator | (h) | (%) | Th•Mn* | Corrected* | Ð |
| 105 | 200/1/4/0.2/1 | 210 | 567 | HVA | 24 | — | — | — | — |
| 115 | 200/1/16/0.2/1 | 210 | 567 | ACAC | 0.5 | 79.1 | 79.3 | 58.5 | 1.13 |
| 104 | 200/1/4/0.2/1 | 210 | 567 | ACAC | 0.5 | 86.5 | 86.7 | 63.2 | 1.15 |
| 107 | 200/1/1/0.2/1 | 210 | 567 | ACAC | 0.5 | 55.6 | 55.6 | 62.5 | 1.27 |
| 111 | 200/1/0.25/0.2/1 | 210 | 567 | ACAC | 1 | 41 | 41 | 35.3 | 1.26 |
| 108 | 200/1/4/0.2/1 | 210 | 270 | ACAC | 1 | 54 | 54 | 75.3 | 1.21 |
| 112 | 200/1/4/0.2/1 | 210 | 1134 | ACAC | 0.5 | 94 | 94 | 70.9 | 1.17 |
| 116 | 200/1/4/0.2/1 | 210 | 105 | ACAC | — | — | — | — | — |
| 106 | 800/1/4/0.8/4 | 210 | 142 | ACAC | 24 | — | — | — | — |
| 110^{α} | 800/1/4/0.8/4 | 210 | 567 | ACAC | 0.25 | 11.1 | 44 | 41.1 | 1.32 |
| 114 | 800/1/16/0.8/4 | 210 | 567 | ACAC | 0.75 | 86.5 | 346 | 85.4 | 1.16 |
| 117 | 800/1/16/0.8/4 | 210 | 2268 | ACAC | NK | 99 | 396 | 107 | 1.2 |
| 118* | 800/1/64/0.8/4 | 210 | 2268 | ACAC | NK | 99 | 396 | 105 | 1.22 |

*Mn expressed in 10⁻³; M = OEOMA₅₀₀, [M] = 10 vol %, in PBS, [Glucose] = 21 mM, [GOx] = 210 nM, RXN temperature = 35° C.; DP 200 [I] = 1 mM, DP 800 [I] = 0.25 mM;
*[GOx] = 840 nM & [Glucose] = 95 mM;
^{α}[Glucose] = 5 mM & [ACAC] = 1 mM; NK = No kinetics; Correction was executed using Mark-Houwink Equation based on PMMA-Toluene as universal calibration.

### Example 4—Oxygen Tolerant Aqueous Photo-ATRP Under Blue Light

A series of aqueous photo-ATRP reactions were conducted under blue light (450 nm) radiation with and initial ratio of reagents, [Glucose]=200 mM, [GOx]=2 μM, [sodium pyruvate]=100 mM; total volume=5 mL, TABLE 12 provides a list of reaction conditions and their results. It can be observed that the polymerizations were well controlled and reached high conversions in less than 2 hours. The “discrepancy” between the theoretical MW and measured MW can be attributed to the difference between the GPC standard and the higher molecular weight biocompatible monomer (PEG₂ₖBPA) employed for the polymerization which additionally formed a brush like molecule known to result in a lower measured MW than the actual MW.

TABLE 12

| Entry | M/I/Cu/TPMA | Degas | Initiator | Solvent | Time/h | Conv./% | M_{n, Th} | M_{n, GPC} | Ð |
|---|---|---|---|---|---|---|---|---|---|
| LF-3-81 | 200/1/0.2/1 | N₂ purge | PEG₂ₖBPA | H₂O + Br | 3.5 | 0 | — | — | — |
| LF-3-82 | 200/1/0.2/1 | Glu + GOx + Pyr | PEG₂ₖBPA | H₂O + Br | 2 | 97 | 98.9 | 58.8 | 1.29 |
| LF-3-83 | 200/1/0.2/0.24 | Glu + GOx + Pyr | PEG₂ₖBPA | H₂O + Br | 2 | 96 | 97.4 | 61.1 | 1.22 |
| LF-3-87 | 200/1/0.2/1 | Glu + GOx + Pyr | PEG₂ₖBPA | 1X PBS | 2 | 96 | 97.3 | 67.3 | 1.09 |
| LF-3-88 | 200/1/0.2/1 | Glu + GOx + Pyr | HO-EBiB | H₂O + Br | 2 | 96 | 96.5 | 46.4 | 1.16 |
| LF-3-89 | 400/1/0.4/2 | Glu + GOx + Pyr | PEG₂ₖBPA | 1X PBS | 2 | 82 | 164.7 | 130.2 | 1.09 |
| LF-3-90 | 100/1/0.1/0.5 | Glu + GOx + Pyr | PEG₂ₖBPA | 1X PBS | 2 | 55 | 29.7 | 32.2 | 1.11 |
| LF-3-91 | 200/1/0.2/1 | Glu + GOx + Pyr | BSA-[iBBr]₃₀ | 1X PBS | 1.5 | 66 | 66.0 | 67.2 | 1.09 |

Choice of solvent and initiator

| Entry | M/I/Cu/TPMA | Degas | Initiator | Solvent | Time/h | Conv./% | M_{n, Th} | M_{n, GPC} | Ð |
|---|---|---|---|---|---|---|---|---|---|
| LF-3-82 | 200/1/0.2/1 | Glu + GOx + Pyr | PEG₂ₖBPA | H₂O + Br | 2 | 97 | 98.9 | 58.8 | 1.29 |
| LF-3-87 | 200/1/0.2/1 | Glu + GOx + Pyr | PEG₂ₖBPA | 1X PBS | 2 | 96 | 97.3 | 67.3 | 1.09 |
| LF-3-88 | 200/1/0.2/1 | Glu + GOx + Pyr | HO-EBiB | H₂O + Br | 2 | 96 | 96.5 | 46.4 | 1.16 |

Effect of various Cu ppm

| Entry | M/I/Cu/TPMA | Degas | Cu ppm | Solvent | Time/h | Conv./% | M_{n, Th} | M_{n, GPC} | Ð |
|---|---|---|---|---|---|---|---|---|---|
| LF-3-89 | 400/1/0.4/2 | Glu + GOx + Pyr | 1000 | 1X PBS | 2 | 82 | 164.7 | 130.2 | 1.09 |
| LF-3-93 | 400/1/0.2/1 | Glu + GOx + Pyr | 500 | 1X PBS | 2 | 96 | 194.2 | 116.0 | 1.09 |
| LF-3-94 | 400/1/0.04/0.2 | Glu + GOx + Pyr | 100 | 1X PBS | 2 | 86 | 173.5 | 112.1 | 1.16 |
| LF-3-95 | 400/1/0.008/0.04 | Glu + GOx + Pyr | 50 | 1X PBS | 2 | 70 | 142.2 | 120.4 | 1.39 |
| LF-3-96 | 400/1/0.04/0.2 | Glu + GOx + Pyr* | 100 | 1X PBS | 2 | 79 | 159.9 | 111.3 | 1.09 |

Effect of various target DPₙ

| Entry | M/I/Cu/TPMA | Degas | Initiator | Solvent | Time/h | Conv./% | M_{n, Tn} | M_{n, GPC} | Ð |
|---|---|---|---|---|---|---|---|---|---|
| LF-3-87 | 200/1/0.2/1 | Glu + GOx + Pyr | PEG₂ₖBPA | 1X PBS | 2 | 96 | 97.3 | 67.3 | 1.09 |
| LF-3-89 | 400/1/0.4/2 | Glu + GOx + Pyr | PEG₂ₖBPA | 1X PBS | 2 | 82 | 164.7 | 130.2 | 1.09 |
| LF-3-90 | 100/1/0.1/0.5 | Glu + GOx + Pyr | PEG₂ₖBPA | 1X PBS | 2 | 55 | 29.7 | 32.2 | 1.11 |
| LF-3-102 | 100/1/0.1/0.5 | Glu + GOx + Pyr | PEG₂ₖBPA | 1X PBS | 2 | 89 | 46.7 | 47.2 | 1.19 |

Effect of lowering [GOx] by half

| Entry | M/I/Cu/TPMA | Degas | [GOx] | Cu ppm | Time/h | Conv./% | M_{n, Th} | M_{n, GPC} | Ð |
|---|---|---|---|---|---|---|---|---|---|
| LF-3-94 | 400/1/0.04/0.2 | Glu + GOx + Pyr | 2 μM | 100 | 2 | 86 | 173.5 | 112.1 | 1.16 |
| LF-3-98 | 400/1/0.04/0.2 | Glu + GOx + Pyr | 1 μM | 100 | 4.5 | 83 | 168.2 | 92.9 | 1.30 |
| LF-3-87 | 200/1/0.2/1 | Glu + GOx + Pyr | 2 μM | 1000 | 2 | 96 | 97.3 | 67.3 | 1.09 |
| LF-3-99 | 200/1/0.2/1 | Glu + GOx + Pyr | 1 μM | 1000 | 4.5 | 92 | 92.2 | 59.4 | 1.21 |

Effect of changing [Na pyruvate]

| Entry | M/I/Cu/TPMA | Na pyruvate | | Cu ppm | Time/h | Conv./% | M_{n, Th} | M_{n, GPC} | Ð |
|---|---|---|---|---|---|---|---|---|---|
| LF-3-87 | 200/1/0.2/1 | 100 | mM | 1000 | 2 | 96 | 97.3 | 67.3 | 1.09 |
| LF-3-100 | 200/1/0.2/1 | 50 | mM | 1000 | 2 | 96 | 97.6 | 69.7 | 1.18 |
| LF-3-101 | 200/1/0.2/1 | 200 | mM | 1000 | 2 | 96 | 98.8 | 70.4 | 1.19 |
| LF-3-104 | 200/1/0.2/1 | 25 | mM | 1000 | 2 | 85 | 85.7 | 59.4 | 1.25 |

Control exp with/without light, and no GOx/pyruvate

| Entry | M/I/Cu/TPMA | Degas | Irradiation | Time/h | Conv./% | M_{n, Th} | M_{n, GPC} | Ð |
|---|---|---|---|---|---|---|---|---|
| LF-3-82 | 200/1/0.2/1 | Glu + GOx + Pyr | Y | 2 | 97 | 98.9 | 58.8 | 1.29 |
| LF-3-82C | 200/1/0.2/1 | Glu + GOx + Pyr | N | 15 | 71 | 73.2 | 38.9 | 1.32 |
| LF-3-86 | 200/1/0.2/1 | +Glu, No degas | Y | — | — | — | — | — |

Grafting from protein

| Entry | M/I/Cu/TPMA | Degas | Initiator | Solvent | Time/h | Conv./% | M_{n, Th} | M_{n, GPC} | Ð |
|---|---|---|---|---|---|---|---|---|---|
| LF-3-91 | 200/1/0.2/1 | Glu + GOx + Pyr | BSA-[iBBr]₃₀ | 1X PBS | 1.5 | 66 | 66.0 | 67.2 | 1.09 |
| LF-3-103 | 200/1/0.2/1 | Glu + GOx + Pyr | BSA-[iBBr]₃₁ | 1X PBS | 2 | 93 | 93.3 | 84.3 | 1.17 |

*incubated in dark for 30 min

### Example 5—Oxygen Fueled ATRP

Starting conditions for the polymerizations employed the following ratio of reagents: M/I/ACAC/GOx/HRP/Cu/TPMA of 200/1/1/0.0002/0.00054/0.2/1 unless a specific variation is described. The effect of ACAC concentration on the rate of polymerization is summarized in TABLE 13. Using lower equivalents of ACAC than the concentration of alkyl bromide initiator ([I]) (Entry 3) caused the reaction to proceed slowly reaching 41% conversion after 1 hour. When the amount of ACAC was equimolar to the [I] (Entry 4), the polymerization proceeded faster reaching 83% conversion in 30 minutes with dispersity similar to that 10 reported for entry 3, Ð=1.27. Increasing the concentration of ACAC to 4× and 16× the [I] (Entries 5-6) increased the rate and reduced dispersity (Ð≤1.15).

TABLE 13
Results for oxygen fueled ATRP under different conditions

| | | HRP | t | kₚ^{app} | | | | |
|---|---|---|---|---|---|---|---|---|
| Entry^{a} | M/I/ACAC | (nM) | (h) | (min⁻¹) | Conversion^{b} | M_{n, th}^{c} | M_{n, GPC}^{d} | M_{w}/Mₙ^{d} |
| 1 | 200/1/0 | 567 | 2.5 | — | — | — | — | — |
| 2 | 200/1/0 | — | 2.5 | — | — | — | — | — |
| 3 | 200/1/0.25 | 567 | 1 | 0.0094 | 41 | 41200 | 35300 | 1.26 |
| 4 | 200/1/1 | 567 | 0.5 | 0.0386 | 82.61 | 82800 | 65250 | 1.27 |
| 5 | 200/1/4 | 567 | 0.5 | 0.0798 | 86.5 | 86700 | 63200 | 1.15 |
| 6 | 200/1/16 | 567 | 0.5 | 0.0983 | 79.1 | 79300 | 58500 | 1.13 |
| 7 | 200/1/4 | 100 | 2.5 | — | — | — | — | — |
| 8 | 200/1/4 | 270 | 0.5 | 0.0323 | 58.1 | 58300 | 38350 | 1.13 |
| 9 | 200/1/4 | 1130 | 0.5 | 0.0951 | 94 | 94200 | 70900 | 1.17 |

^{a}Reaction conditions: M = OEOMA500, [M] = 210 mM (10 vol %) in PBS, pH = 6, [Cu] = 1000 ppm, [Glucose] = 21 mM, [GOx] = 210 nM, RXN temperature = 37° C.; [I] = 1 mM;
^{b}Conversion determined by 1H NMR.
^{c}Calculated on the basis of conversion (i.e., Mn, th = BPAA + [OEOMA500]0/[BPA]0 × conversion × Mn of monomer).
^{d}Determined by GPC in THF, using Mark-Houwink parameters.

Interestingly, although the difference in ACAC concentration between entries 5 and 6 is fourfold, the results obtained for the polymerization were very similar, which could indicate that the saturation limit of HRP under these conditions was reached. Consequently, the concentration of HRP was varied (Entries 5, 7-9). The changes in concentration can be more easily visualized for the comparison against GOx at concentration 210 nM. Therefore, a concentration of HRP less than half of GOx (100 nM, Entry 7) was not sufficient to consume the hydrogen peroxide present in the solution and create a steady flow of radicals to generate Cu(I) activators. By increasing the concentration of HRP to 270 nM (1.3×[GOx]) (Entry 8), conversion reached 58.1% in 30 minutes. However, by increasing the concentration of HRP to 567 nM (2.7×[GOx], (Entry 5) and 1130 nM (5.4×[GOx], (Entry 9), conversion reached 86.5% and 94%, respectively in 30 minutes. While plausible Fenton-like reactions may not be completely inhibited due to a lag in the catalytic activity of HRP, the experiments conducted in a completely open flask were fast and showed good correlation between theoretical Mₙ and experimental values and low values of M_{w}/Mₙ. Control experiments (Entries 1-2) demonstrated that addition of both ACAC and HRP was necessary for the polymerization.

Once that the core parameters in this enzymatic machinery were adjusted, (TABLE 14) the limits of the control provided by Cu^{II}/TPMA complex at lower ppm concentrations ([Cu]) versus monomer was studied. The results revealed a correlation between the amount of Cu and the M_{w}/Mₙ. Higher concentrations of Cu yielded better control and provided lower dispersities, while lower concentrations showed higher M_{w}/Mₙ and a lower rate of reaction, as expected for ATRP. It is important to highlight that using only 100 ppm of catalyst (Entry 12) a polymer with Mₙ=33700 and Ð=1.31 in was obtained in 30 minutes. These numbers are significant, considering that the actual concentration of Cu in a 5 mL reaction mixture is 10 ppm, which approaches the concentration of Cu in drinking water.

TABLE 14
Results of aqueous oxygen fueled ATRP in the presence of
various concentrations of Cu and targeting different DPₙ

| | | Cu | t | kₚ^{app} | | | | |
|---|---|---|---|---|---|---|---|---|
| Entry^{a} | M/I/Cu/TPMA | (ppm) | (min) | (min⁻¹) | Conversion^{b} | M_{n, th}^{c} | M_{n, GPC}^{d} | M_{w}/Mₙ^{d} |
| 5 | 200/1/0.2/1 | 1000 | 30 | 0.0798 | 86.5% | 86700 | 63200 | 1.15 |
| 10 | 200/1/0.1/0.5 | 500 | 60 | 0.0408 | 81.6% | 81800 | 58600 | 1.25 |
| 11 | 200/1/0.06/0.3 | 300 | 60 | 0.0291 | 70.3% | 70500 | 49000 | 1.27 |
| 12 | 200/1/0.02/0.1 | 100 | 60 | 0.0144 | 37% | 37200 | 33700 | 1.31 |
| 13 | 50/1/0.005/0.025 | 1000 | 30 | 0.0736 | 77.9% | 19700 | 20500 | 1.14 |
| 14 | 100/1/0.01/0.05 | 1000 | 30 | 0.0876 | 81.6% | 41000 | 29400 | 1.13 |
| 15 | 150/1/0.015/0.075 | 1000 | 30 | 0.0588 | 71.8% | 54000 | 33360 | 1.15 |

^{a}Reaction conditions: M = OEOMA₅₀₀, [M] = 210 mM (10 vol %) in PBS, pH = 6, [Cu] = 1000 ppm, [Glucose] = 21 mM, [GOx] = 210 nM, RXN temperature = 37° C.; [I] = 1 mM;
^{b}Conversion determined by ¹H NMR.
^{c}Calculated on the basis of conversion (i.e., M_{n, th} = BPAA + [OEOMA₅₀₀]₀/[BPA]₀ × conversion × Mₙ of monomer).
^{d}Determined by GPC in THF, using Mark-Houwink parameters.

### Example 6—GOx Self-Deoxygenated ATRP

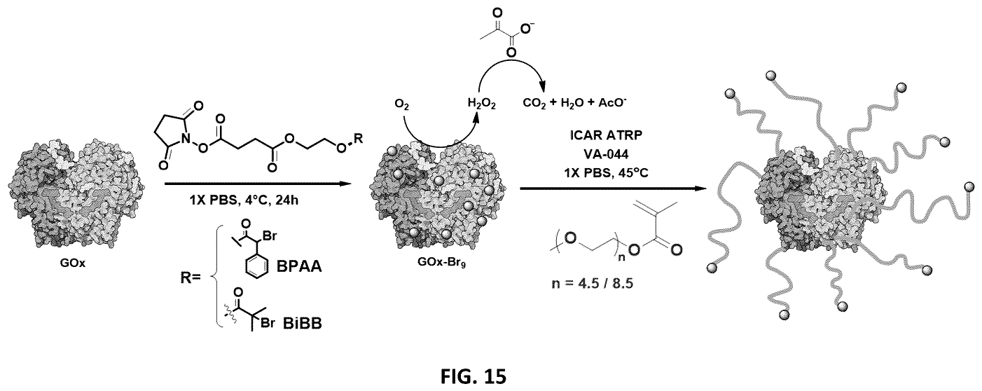

A series of Self-Deoxygenated ATRP reactions were conducted using the ATRP initiator-modified GOx. As illustrated in FIG. 15, native GOx was first modified with two versions of ATRP initiator, and then the modified GOx catalyzed deoxygenation while polymers were continuously grafted from GOx. TABLE 15 provides a list of reaction conditions of the two types of initiators and modified sites. The reaction conversion reached 70˜93% within 10 minutes, yielding giving polymers with low dispersity (1.15˜1.16) that were analyzed by cleaving the polymer by reaction with 5% NaOH solution for 2 hours.

### Example 7—Reversible Immobilization of GOx onto Solid Surfaces

Immobilized enzymes typically are more robust and more resistant to environmental changes than free enzymes in solution. More importantly, the heterogeneity of an immobilized enzyme system can allow for easy recovery of enzymes and products, multiple reuses of enzymes, continuous manipulation of enzymatic processes, rapid termination of reactions, and a wider variety of bioreactor designs. In the present case, GOx was immobilized onto glassware or another solid support, permitting the GOx to be separated and reused, thus simplifying the reaction setup.

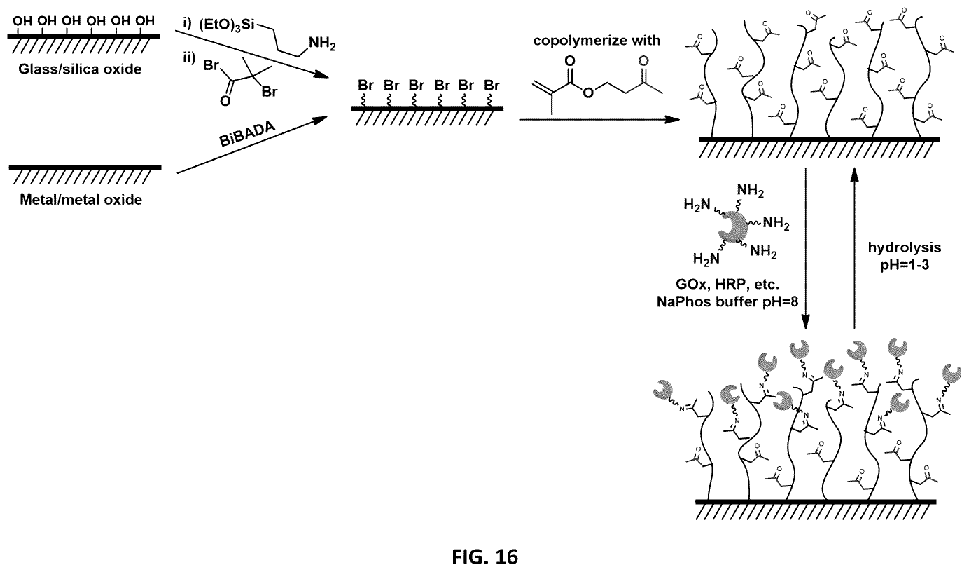

A general scheme illustrating the immobilization of GOx is illustrated in FIG. 16. A solid or material surface was functionalized with an ATRP initiator (e.g., 3-(triethoxysilyl) propylamine and α-bromoisobutyryl bromide for glass/silica oxide surfaces, or by direct anchoring with 12-(2-bromoisobutyramido) dodecanoic acid (BiBADA) for metal/metal oxide surfaces). Polymers were then grafted from the material surfaces. When the grafted copolymer contained a fraction of acetylethyl methacrylate (AEMA, a novel ketone containing monomer), GOx was attached to the material-polymer surface via lysine residues, through formation of an imine in the presence of a phosphate buffer under pH 8. The immobilization of GOx was reversible due to the nature of the imine linkage, and was hydrolysable under strong acidic conditions (pH 1-3). Ketone functionality could be restored, thereby reforming surfaces that were ready to be renewed for another batch of enzymes. It is noted that such immobilization also can be conducted for other enzymes (e.g., P2Ox).

TABLE 15
Results of GOx-initiator self-deoxygenated ATRP

| | | Initiator | # initiators | RXN time | Conv. | M_{n, Th} × | M_{n, Corr} × | |
|---|---|---|---|---|---|---|---|---|
| Entry | M/I/VA-44/Cu/Ligand | type | on Lys | (min) | (%) | 10⁻³ | 10⁻³ | Ð |
| 1 | 400/1/0.3/0.2/1 | BPAA | 9 | 10 | 77 | 185 | 89.2 | 1.15 |
| 2 | 400/1/0.3/0.2/1 | BiBB | 7 | 10 | 92.5 | 154 | 60.9 | 1.16 |

M = OEOMA₅₀₀, [M] = 10 vol % in PBS, [NaBr] = 100 mM, [Glucose] = 200 mM, [Sodium Pyruvate] = 100 mM, [GOx] = 2 μM, RXN temperature = 45° C.; Determined by GPC in THF, using Mark-Houwink parameters

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for polymerizing free radically copolymerizable monomers, comprising combining:
(a) a deoxygenated polymerization medium comprising (i) a transition metal catalyst or metal-free organic complex that can participate in a redox reaction, and (ii) an initiator comprising one or more redox transferable atoms or groups;
(b) an oxygen scavenger; and
(c) a reactive oxygen species (ROS) scavenger.

2. The method of claim 1, wherein the polymerization medium is an aqueous medium.

3. The method of claim 1, wherein the method is carried out in a reactor with a limited head space comprising air.

4. The method of claim 1, wherein the method is carried out in a reactor open to the atmosphere.

5. The method of claim 1, wherein the oxygen scavenger comprises glucose and glucose oxidase (GOx).

6. The method of claim 1, wherein the ROS scavenger comprises one or more of pyruvate, horseradish peroxidase, an α-keto-acid, catalase, and a catalase-like enzyme selected from the group consisting of ascorbate peroxidase, cytochrome C peroxidase, haloperoxidase, hemoprotein, glutathione peroxidase, glucose oxidase, laccase, monoamine oxidase, cytochrome P450 oxidase, NADPH oxidase, xanthine oxidase, L-gulonolactone oxidase, and superoxide dismutase.

7. The method of claim 1, wherein the oxygen scavenger comprises glucose and GOx, and wherein the ROS scavenger comprises one or more of pyruvate, horseradish peroxidase, an α-keto-acid, catalase, and a catalase-like enzyme selected from the group consisting of ascorbate peroxidase, cytochrome C peroxidase, haloperoxidase, hemoprotein, glutathione peroxidase, glucose oxidase, laccase, monoamine oxidase, cytochrome P450 oxidase, NADPH oxidase, xanthine oxidase, L-gulonolactone oxidase, and superoxide dismutase.

8. The method of claim 1, wherein the initiator is a bioresponsive molecule with one or more site specific functional initiators.

9. The method of claim 8, wherein the bio-responsive molecule comprises a protein, polypeptide, polynucleotide, aptamer, nucleic acid, or other biomolecule that is incorporated into the formed conjugate with the polymer grown from the site-specific incorporated initiator site(s).

10. The method of claim 1, comprising transition metal-mediated controlled polymerizing of vinyl monomers in an aqueous based polymerization medium.

11. The method of claim 1, wherein the method is an Initiators for Continuous Activator Regeneration (ICAR) atom transfer radical polymerization (ATRP), and wherein a free radical source is generated in situ by reaction of an enzyme with the product of the oxygen scavenging reaction.

12. The method of claim 1, wherein the method is a photo-ATRP, wherein a free radical source is generated in situ by photo-based reaction of a reducing agent, and wherein oxygen is removed by reaction of an enzyme with the product of the oxygen scavenging reaction.

13. The method of claim 1, wherein the method is an activator regenerated by electron transfer (ARGET) ATRP, wherein a free radical source is generated in situ by reaction of a reducing agent, and wherein oxygen is removed by reaction of an enzyme with the product of the oxygen scavenging reaction.

14. (canceled)

15. The method of claim 1, wherein the oxygen scavenger comprises glucose and GOx, and wherein the GOx is immobilized on a solid surface.

16. (canceled)

17. An oxygen-driven ATRP method, wherein the method comprises combining:
(a) a deoxygenated polymerization medium comprising (i) copper at a concentration less than 1500 ppm, wherein the copper is capable of participating in a redox reaction, and (ii) a halogen-containing initiator comprising one or more redox transferable atoms or groups;
(b) an oxygen scavenger comprising glucose and GOx; and
(c) a ROS scavenger comprising a pyruvate, horseradish peroxidase, an α-keto-acid, a catalase, or a catalase-like enzyme,
wherein the concentrations of the oxygen scavenger and the ROS scavenger are sufficient to provide a continuous source of radicals for controlled polymerization from the halogen-containing initiator.

18. The method of claim 17, wherein the polymerization medium is an aqueous medium.

19. The method of claim 17, wherein the method is carried out in a reactor with a limited head space comprising air.

20. The method of claim 17, wherein the method is carried out in a reactor open to the atmosphere.

21. The method of claim 17, wherein the ROS scavenger comprises a catalase-like enzyme selected from the group consisting of ascorbate peroxidase, cytochrome C peroxidase, haloperoxidase, hemoprotein, glutathione peroxidase, glucose oxidase, laccase, monoamine oxidase, cytochrome P450 oxidase, NADPH oxidase, xanthine oxidase, L-gulonolactone oxidase, and superoxide dismutase.

22. The method of claim 17, GOx, and wherein the GOx is immobilized on a solid surface.

23. (canceled)
